(19) United States
(12) Patent Application Publication
(10) Pub. No.: US 2019/0091256 A1
(43) Pub. Date: Mar. 28, 2019

(54) THERAPEUTIC TARGETS FOR LIN-28-EXPRESSING CANCERS

(71) Applicants: Beth Israel Deaconess Medical Center, Boston, MA (US); Yale University, New Haven, CT (US)

(72) Inventors: Frank SLACK, Waban, MA (US); Giovanni STEFANI, New Haven, CT (US); Xiaowei CHEN, New Haven, CT (US)

(21) Appl. No.: 16/081,741

(22) PCT Filed: Mar. 1, 2017
(86) PCT No.: PCT/US2017/020144

§ 371 Date: Aug. 31, 2018

Related U.S. Application Data
(60) Provisional application No. 62/302,285, filed on Mar. 2, 2016.

(51) Int. Cl. A61K 31/7105; C12N 15/11; C07K 1/18

(52) U.S. Cl. CPC A61K 31/7105; C12N 15/111; C07K 1/18; C12Q 2600/136; C12Q 2600/112; C12N 2320/31; C12Q 2521/101; A01K 2267/0331; C12Q 2600/178; C12N 2310/11; C12N 2320/10; C12Q 2600/106

(57) ABSTRACT

The present disclosure identifies RNAs (including mRNAs and miRNAs) that are bound by LIN-28 in C. elegans. Many of these RNAs have clear human orthologs, and many of these human orthologs are common druggable targets in cancer and/or other diseases, such as kinases, phosphatases, methyltransferases, phosphodiesterases, etc. Accordingly, the present disclosure provides biological targets for LIN-28 expressing cancers, and which are thus useful for selecting chemical and/or biological agents for cancer treatment.

### PRIORITY

This application claims the benefit of, and claims priority to, U.S. Provisional Application No. 62/302,285, filed Mar. 2, 2016, which is hereby incorporated by reference in its entirety.

### FEDERAL FUNDING

The invention was made with U.S. government support under NIH grant number AG033921. The U.S. government has certain rights in the invention.

### FIELD OF THE INVENTION

The present invention relates, in part, to biological targets for LIN-28 expressing cancers. The invention further provides chemical and/or biological agents for treating LIN-28 expressing cancers.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BID-004PC_Sequence Listing; date recorded: Mar. 1, 2017; file size: 4.1 kb).

### BACKGROUND

The evolutionarily conserved gene lin-28 encodes an RNA-binding protein, LIN-28, and is an important regulator of the proper temporal succession of several developmental events in both invertebrates and vertebrates. lin-28 interacts genetically with other heterochronic genes: the persistent expression of lin-14 requires LIN-28, while the lin-28 mutant phenotype can be suppressed by mutations in lin-46 (Arasu et al. 1991) (Pepper et al. 2004). Furthermore, mutation of let-7 partially rescues the precocious differentiation of seam cells in lin-28 mutants, and lin-28 is required for the correct temporal expression of let-7 (Reinhart et al. 2000) (Johnson et al. 2003) (Van Wynsberghe et al. 2011).

At the cellular and organismal level, the LIN-28 protein promotes stemness and proliferation, and inhibits differentiation. In addition, the functions and pattern of expression of lin-28 are, in broad terms, consistent between C. elegans and vertebrates. Furthermore, LIN-28 affects glucose metabolism as documented in genetically modified mice (Zhu et al. 2011). The functional proprieties of LIN-28 have been exploited for the induction of pluripotency in human fibroblasts, by the simultaneous transduction of LIN28, OCT4, SOX2 and NANOG (Yu et al. 2007). Moreover, the proliferative and anti-differentiation functions of LIN-28 are co-opted in a number of human cancers, where its expression is re-activated, resulting in more aggressive and rapidly growing tumors (Viswanathan et al. 2009). Expression of LIN-28 is linked to cancer prognosis.

LIN-28 includes at least two functional domains: a cold shock domain (CSD) and two CCHC-type zinc-finger (ZnF) domains, both well-known nucleic acid recognition motifs. For example, in vertebrates, LIN-28 inhibits the maturation of the miRNA let-7, possibly through the binding to sequences in the terminal loop of pri or pre-/et-7 in mammals (Piskounova et al. 2008) (Viswanathan et al. 2008) (Newman et al. 2008) (Heo et al. 2008) (Rybak et al. 2008).

While forward genetics has positioned lin-28 in the heterochronic pathway and studies in cells in culture have revealed interactions of LIN-28 with a number of mRNAs (Wilbert et al. 2012) (Cho et al. 2012), (Hafner et al. 2013), the molecular characterization of LIN-28 function in the context of development and abnormal tissue growth, such as cancer, is being elucidated.

### SUMMARY OF THE INVENTION

The present disclosure identifies RNAs (including mRNAs and miRNAs) that are bound by LIN-28 in C. elegans. Many of these RNAs have clear human orthologs, and many of these human orthologs are common druggable targets in cancer and/or other diseases, such as kinases, phosphatases, methyltransferases, phosphodiesterases, etc. Accordingly, the present disclosure provides biological targets for LIN-28 expressing cancers, including protein and polynucleotide targets, and which are thus useful for selecting chemical and/or biological agents for cancer treatment.

The present invention in various aspects and embodiments provides a method of identifying an agent for treating LIN-28-expressing cancer. The method comprises providing a LIN-28 target, and selecting an agent to modulate the expression or activity of the LIN-28 target. LIN-28 targets shown in Table 1, which are human orthologs of transcripts bound by LIN-28 in C. elegans. Additional polynucleotide motifs targeted by LIN-28, such as the motif GGAG and biological targets that comprise this motif, are described herein. LIN-28 impacts biological targets that represent several promising classes of drug targets such as kinases, phosphatases, methyltransferases, transcription factors, methylases/acetylases, polymerases, proteases, phosphodiesterases, and further impacts targets in the mTOR and MAP Kinase pathways and other pathways related to cancer biology, thereby opening numerous avenues for impacting cancers characterized by LIN-28 expression or activation.

Activity against the LIN-28 target or LIN-28 expressing cancer can be confirmed by in vitro assay, animal model relevant to LIN-28-expressing cancer, and/or clinically in patients. In selecting an active agent, a panel or library of candidate agents may be tested against the target in a screen, including a high throughput screen, or tested for an ability to activate or inhibit a pathway (e.g., a cell signaling pathway) that comprises the LIN-28 target. Exemplary assays are described herein for testing candidate agents for activity against LIN-28 expressing cancers.

In other aspects, the invention provides companion diagnostic assays for cancer treatment. Specifically, the invention allows cancer biopsies to be tested for LIN-28 or LIN-28 target expression or activity, so that candidate agents (including those identified by the methods described herein) can be appropriately selected for treatment on a personalized basis.

Other aspects and embodiments of the invention will be apparent from the following detailed description.

### BRIEF DESCRIPTION OF THE DRAWINGS

FIG. 1: (Panel A) A genome-wide view of LIN-28 interactions with the C. elegans transcriptome. Reads from a representative CLIP experiment, a matching background control and an Input (RNAseq) control are displayed in Integrated Genome Viewer (IGV) (Robinson et al. 2011). Number of reads in each line was normalized by total number of mapped reads. (Panel B) Reproducibility of two CLIP experimental trials. (Panel C) Correlation between read depth in CLIP samples and RNA abundance (RNAseq). (Panel D) Motif discovered by the Multiple EM for Motif Elicitation (MEME) tool within the binding sites dataset defined by peak analysis. (Panel E) Motif discovered by MEME analysis within the binding sites dataset defined by deletions. (Panel F) Motif discovered by MEME analysis within the binding sites dataset defined by insertions. (G) Motifs discovered by MEME analysis within the binding sites dataset defined by substitutions.

FIG. 2: (Panel A) LIN-28 binding site distribution within mRNA regions. The X-axis is the position between 200 bp upstream of start codons and 750 bp downstream of stop codons. The highest enrichment of LIN-28 binding sites is observed within 3′ UTRs. (Panel B) Gene Ontology enrichment analysis for LIN-28 bona fide targets; top seven scoring clusters are shown. Clusters were defined using DAVID Gene Functional Annotation Clustering. GO BP (biological processes) ‘FAT’ annotations and ‘highest’ stringency were used. Clusters are annotated with representative GO terms and corresponding Benjamini-Hochberg FDR corrected P values, and ranked by enrichment score.

FIG. 3: LIN-28 interacts with heterochronic genes mRNAs. (Panel A) Map of LIN-28 interactions with the lin-14 mRNA visualized by IGV. The number of reads in each track was normalized by the total number of mapped reads. (Panel B) RNA-co-immunoprecipitated with LIN-28 was analyzed by RT-qPCR with primers for hsp-12.2 (negative control) and lin-14. The abundance of these mRNAs in the RIP sample were normalized to their abundance in the input material. (Panel C) The abundance of lin-14 in wild type animals (N2) and lin-28 mutants, detected by qPCR. (Panel D) Map of LIN-28 interactions with lin-46 mRNA. (Panel E) Map of LIN-28 interactions with kin-20 mRNA. (Panel F) Map of LIN-28 interactions with din-1 mRNA. (Panel G) RIP analysis of interactions between LIN-28 and hsp-12.2 (negative control), din-1, egl-30 mRNAs and let-7 primary transcript (pri-let-7).

FIG. 4: LIN-28 interactions with pri-let-7. (Panel A) Map of LIN-28 interactions with let-7 precursors visualized by IGV. The number of reads in each line was normalized by the total number of mapped reads. Pre-let-7, pri-let-7 and a transgene capable of rescuing the let-7 mn112 and mg279 mutations are shown in the lower tracks (Reinhart et al. 2000). Since pri-let-7 is transcribed from the minus strand, its 5′ end corresponds to the right hand end of the bar, while its 3′ end to the left. (Panel B) The secondary structures of pre-let-7 and LIN-28 binding site (LBS) predicted using the mfold algorithm, superimposed to a schematic representation of pri-let-7, the pre-let-7 and pri-let-7 tracks, and a bar graph representation of the number of reads obtained by LIN-28 HITS-CLIP. For ease of representation, shown is a schematic drawing of pri-let-7 with annotation tracks and bar graph flipped horizontally compared to Panel A, so that the 5′ end in on the left side, while the 3′ end is on the right side.

FIG. 5: Binding of LIN-28 to pri-let-7 assessed through an in vitro UV-crosslinking assay with radiolabeled RNA. (Panel A) Autoradiography showing LIN-28 (fused to GFP, HA and flag, migrating in SDS-PAGE at around 55 kDa), expressed in C. elegans larvae, immuno-precipitated and UV cross-linked to the indicated P32 body-labeled RNAs. The same filter used for radiography was probed with antibody against HA to verify the presence of equal amounts of LIN-28 (‘Western Blot’). Labeled RNA corresponding to pre-let-7, LBS and negative control were analyzed by TBE-Urea gel electrophoresis to verify the presence equal amount of probe and its integrity (‘RNA input control’). The panel on the right shows a quantitation of the autoradiography by Phosphoimager. (Panel B) Interaction of LIN-28 with the LBS or a mutated version of it in which GGAG motifs are changed to CCTC. An in vitro UV-crosslinking assay as in Panel A is shown, in which the probe was LBS containing either wild type GGAG motifs (right three lanes) or mutated CTCC (left three lanes). The experiment was executed in triplicate for each probe. In the second and third lane of each probe, cold competitor corresponding to negative control (as in Panel A) was also included in a 40 and 200 folds molar excess compared to the labeled probe. The same filter used for radiography was probed with antibody against HA (‘Western Blot’). Labeled RNA corresponding to GGAG or CTCC probes were analyzed by TBE-Urea gel electrophoresis (‘RNA input control’). The panel on the right shows a quantitation of the autoradiography by Phosphoimager.

FIG. 6: The LBS is required for normal regulation of maturation of let-7 by LIN-28. (Panel A) Schematic representation of the normal pattern of expression of pri-let-7 (blue), LIN-28 (green), pre-let-7 and mature let-7 (orange) during larval development. (Panel B) Mature let-7 levels detected by RT-qPCR at the time of L1 larval molt in transgenic worms carrying a wild type let-7 transgene (WT) or one in which the LBS is deleted (MUT). (Panel C) Mature let-7 levels detected by RT-qPCR at the indicated time points (x axis) in transgenic animals carrying wild type or mutated transgenes as in Panel B.

FIG. 7: Conservation of the GGAG motifs within the LBS and pre-let-7 across species. (Panel A) Alignment of the LBS region of four nematode species (C. elegans, C. remanei, C. briggsae, C. brenneri). (Panel B) Phylogenetic distribution of the let-7 miRNAs in metazoans. For each indicated species, the number of let-7 genes is indicated in the left column (black font). The number of let-7 genes that have GGAG motifs in their precursor's terminal loop is indicated in the middle column (Blue font, ‘GGAG+’), while the number of let-7 genes that lack such feature is indicated in the right column (red font, ‘GGAG−’). The presence of one or two LIN-28 orthologs (Panels A and B) is indicated in the rightmost column

FIG. 8: CIMS analysis. (Panel A) Motifs discovered by MEME analysis within the binding sites dataset defined by deletions, insertions and substitutions within the CLIP2 dataset alone. (Panel B) Deletions (D), substitutions (S) and Insertion (I) were ranked by a binomial test (see Methods), and the presence of the GGAG motif within a stretch of 30 residues surrounding the point mutation was assessed by MEME analysis. The ratio of such sequences containing a GGAG motif is plotted as a function of the mutation ranking. (Panel C) Distribution of the distance of GGAG motif from the peak window center. (Panel D) Distribution of the distance of the GGAG motif from the CIMS (deletion).

FIG. 9: LIN-28 interactions with miR-229 and miR-48. (Panel A) Map of LIN-28 interactions with the miR-229, miR-64, 65, 66 cluster visualized by IGV. Number of reads in each line was normalized by total number of mapped reads. (Panel B) Map of LIN-28 interactions with the miR-48, miR-241 cluster visualized by IGV.

FIG. 10: Binding of LIN-28 to LBS is inhibited in the presence of cold competitor RNA. (Panel A) Autoradiography showing LIN-28 immuno-purified and cross-linked to P32 body-labeled wild type extended LBS RNA as in FIG. 5, panel B. Labeled wild type LBS RNA was cross-linked to LIN-28 in the presence of increasing amounts of cold WT (GGAG) or mutant (CTCC) RNA competitor (molar ratio cold to labeled RNA: 0, 40, 200). The same filter used for radiography was probed with antibody against HA to verify the presence of equal amounts of LIN-28 (‘Western Blot’). (Panel B) Cold competitor RNA (‘scrambled’ used in experiment shown in FIG. 5, panel B) were analyzed on a TBE-Urea polyacrylamide gel to verify the presence of equal amount of probe and its integrity.

FIG. 11: Quantification of the transgene copy number in the transgenic lines used to assay the effects of LBS deletion on let-7 maturation. Transgenic lines were generated by bombardment in unc-119 background. Four stable lines were obtained with the unaltered construct (171.1, 171.2, 171.8, 171.9) and five with the LBS deletion (172.1, 172.3, 172.4, 172.5, 172.6). The copy number of transgene was quantified by qPCR on genomic DNA, using wild type animals for normalization (N2). Lines 171.8, 172.1 and 172.5 were used for experiments.

FIG. 12: Abundance of mature let-7 detected by Taqman qPCR in transgenic animals carrying a WT pri-let-7 transgene (WT) or one in which the LBS was deleted (MUT). In both these transgenic lines, the endogenous let-7 gene was still present. RNA was extracted 12 hours after hatching and the abundance of mature let-7 and pri-let-7 were assessed by Taqman qPCR and SYBR green qPCR, respectively. Bars represent fold change of let-7 abundance in Lin-28 RNAi relative to negative control RNAi, normalized for pri-let-7 abundance.

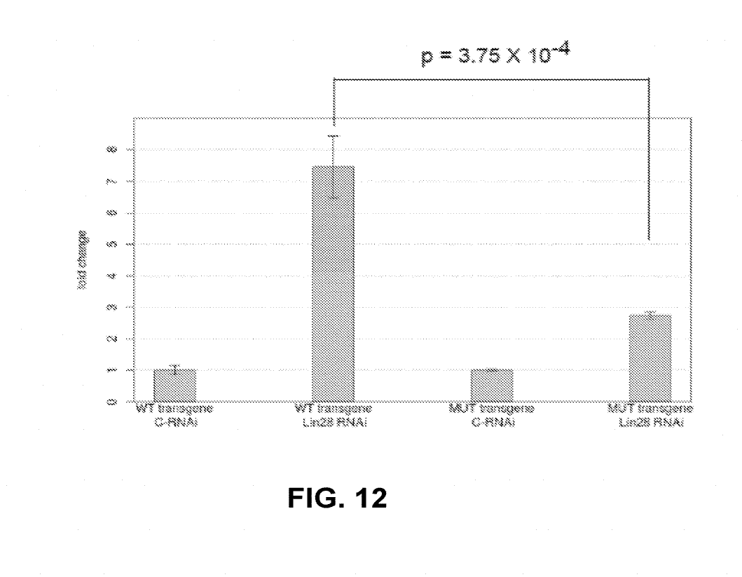

FIG. 13: Conservation of the LBS among different species: (Panel A) Hypothetic secondary structure of LBS region in C. elegans, C. remanei. C. briggsae and C. brenneri (SEQ ID NOs: 12-15). (Panel B) Homo sapiens pri-let-7a3 does not have GGAG motifs in the terminal loop of the precursor (left side of the figure)(SEQ ID NO: 16), and, similarly to C. elegans, has a folded structure about 170 nucleotides downstream, with three GGAG motifs (SEQ ID NO: 17).

FIG. 14: Overlap between the LIN-28 CLIP analysis and a set of known let-7 suppressors (top) and enhancers (bottom).

FIG. 15: qRT-PCR analysis of the genes showing altered expression levels in lin-28(lf) and (lin-28 dLCE gf) mutants animals.

### DETAILED DESCRIPTION OF THE INVENTION

The present invention in various aspects and embodiments provides a method of identifying an agent for treating LIN-28-expressing cancer. The method comprises providing a LIN-28 target, and selecting an agent to modulate the expression or activity of the LIN-28 target. In various embodiments, the LIN-28 target may be selected from the genes or gene fragments in Table 1. Specifically, Table 1 provides human orthologs for LIN-28 targets identified in C. elegans. As discussed further below, these human orthologs include kinases, phosphatases, methyltransferases, transcription factors, methylases/acetylases, polymerases, proteases, phosphodiesterases, among other molecular classes, allowing for active agents to be tested for their potential utility in treating LIN-28-expressing cancers through well-known molecular or cellular assays.

In some embodiments, a LIN-28 target (e.g., a target from Table 1) is selected based on an initial screen. For example, a cell line that requires LIN-28 for growth is provided or created, and one or more targets from Table 1 are silenced (e.g, using siRNA) in the cell line, to thereby identify a LIN-28 target that is required for LIN-28-dependent cell growth. The cell line can be identified in some embodiments from commercially available cell lines, by evaluating LIN-28 expression. Alternatively, or in addition, activity of let-7 in the cell line can be monitored, for example, using a let-7 sensor or reporter according to known methods. In this manner, LIN-28 targets that impact let-7 activity can be identified, including LIN-28 targets whose inhibition might restore let-7 activity. In some embodiments, at least 10 targets from Table 1 are evaluated for their impact on LIN-28-dependent cell growth or impact on let-7 activity. In some embodiments, at least about 20, or at least about 30, or at least about 40, or at least about 50, or at least about 75, or at least about 100 targets from Table 1 are evaluated for their impact on LIN-28-dependent cell growth or impact on let-7 activity. In some embodiments, the targets are evaluated using small interfering RNAs to inhibit their expression in the cell line.

The candidate agent may be a stimulator, inhibitor, agonist or antagonist that affects the expression and/or activity of the LIN-28 target, or a cell pathway that comprises the LIN-28 target. That is, the candidate agent may interact and impact the activity of the LIN-28 target directly (e.g., through direct interaction), or may impact (by inhibition or activation) the cell pathway that comprises the LIN-28 target. In these embodiments, the candidate agent may not interact with LIN-28 directly, but influences the LIN-28 target through another component of the cell pathway. As used herein, cell pathways are defined as in KEGG pathways database: world wide web.genome.jp/kegg/pathway.html, and such pathways are hereby incorporated by reference. Exemplary pathways include mTOR and MAP Kinase pathways.

In some embodiments, a LIN-28 target is identified (from Table 1), a cell pathway that comprises the target is identified or selected, and candidate agents are screened for those that inhibit or activate the pathway. Inhibitors and/or activators of the pathway can further be tested in one or more cell proliferation assays, animal models for LIN-28-expressing cancer, or other model, to validate or confirm the activity of the candidate agent. In some embodiments, candidate agents are tested for their ability to inhibit or activate one or more of mTOR and MAP Kinase pathways.

In some embodiments, a library of candidate agents may be screened against the target in a molecular or cellular assay, or screened against the pathway that comprises the target in a cellular assay. Screening is conducted in a high throughout manner in some embodiments. Based on the target selected, and its biological class and/or involvement in a cellular pathway, candidate agents may be selected based on known activities in some embodiments. For example, candidate agents can include, cellular receptor agonists, partial agonists, or inhibitors (e.g., growth factor receptor inhibitors, or agonists or antagonists for G-Protein Coupled Receptors) for pathways that comprise a LIN-28 target, known kinase inhibitors (e.g., receptor tyrosine kinase inhibitors), known methyltransferase inhibitors, or known phosphodiesterase inhibitors, known phosphatase inhibitors, or candidate agents known to have activity against some cancers. In this manner, promising agents can be screened and optionally derivatized particularly for their potential in treating LIN-28 expressing cancer.

The candidate agent may be any molecule that is known or suspected to modulate a LIN-28 target expression and/or activity. For example, the candidate agent may be an antisense polynucleotide, a small molecule inhibitor or agonist, an antibody or antigen-binding portion thereof (including an antibody or antigen-binding portion thereof against a cellular receptor), a microRNA or microRNA mimic, or small interfering RNA (siRNA). In some embodiments, the candidate agent is an antisense polynucleotide that competes for binding with LIN-28 target RNAs. Exemplary LIN-28 target motifs are described herein. In some embodiments, the candidate agent comprises the motif GGAG, or comprises from 2 to 50 or from 2 to 10 copies of the motif. As shown herein, LIN-28 binds the motif GGAG, and thus, this motif may compete for LIN-28 binding. Alternatively, the candidate agent may have the motif CTCC, or several copies of the motif CTCC (e.g., from 2 to 50 or 2 to 10 copies), so as to block LIN-28 target binding.

Candidate antisense agents can be tested in a molecular or cellular assay for LIN-28 target binding, including an assessment of the impact of off-target binding.

In still other embodiments, the candidate agent is a miRNA or miRNA mimic, for a let-7 family member (e.g., let-7, miR-48, or miR-241), or for miR-229 family member. Such agents can be tested for their ability to inhibit or reverse a cellular phenotype associated with LIN-28 expression.

A variety of molecular assays for the expression and/or activity of the LIN-28 target may be employed. For example, changes in expression of the LIN-28 target may be examined with immunochemical assays such as immunofluorescence, ELISA and Western blot assays, high throughput chip assays such as micro and macro arrays, Northern or Southern blot assays, TaqMan® Probe-Based Gene Expression assay, in situ hybridization assay, or RT-PCR and/or DNA sequencing. For example, if the LIN-28 target is CDK17 kinase, an ELISA may be used to monitor CDK17 protein expression, and/or TaqMan® assay may be used to examine the expression of the polynucleotide encoding the CDK17 protein. The activities of the LIN-28 target may be examined with assays that evaluate the enzyme (or other) activity or activity of the pathway that comprises the LIN-28 target. In some embodiments, the candidate agents are assayed for modulation of expression or abundance of the LIN-28 target in a cell. In some embodiments, the candidate agent is derivatized, and tested for enhanced activity against the LIN-28 target in vitro or in vivo.

In some embodiments, the LIN-28 target is a kinase. For example, the LIN-28 target may be CDK11A, CDK17, NUAK1, NLK, PCK2, CSK, MAP4K1, DMPK, PTP5K1B, HTPK3, CAMKK2, RTOK1, GRK4, TTBK2, ADCK2, CSNK1D/E, ABL2, CASK, UHMK1, DCLK3WNK3, DAPK1, or TLK1, which correspond to human orthologs of LIN-28 targets identified in Table 1.

The candidate agent may be an agonist or inhibitor of a kinase or receptor tyrosine kinase. In some embodiments, the candidate agents include known receptor tyrosine kinase agonists or partial agonists or inhibitors. In some embodiments, the candidate agents include kinase inhibitors. Commercially available kinase inhibitor libraries may be used, and examples include kinase inhibitor library from Selleckchem, Inc. (Houston, Tex., USA), Cayman® kinase screening library from Cayman Chemical, Inc. of Ann Arbor, Mich., USA, and SCREEN-WELL® Kinase Inhibitor library (Enzo Biochem, Inc. Farmingdale, N.Y., USA).

In some embodiments, the candidate agents are tested for modulation of the activity of the target, or a cell pathway comprising the target, using a kinase assay. When testing candidate agents, such as a library of kinase inhibitors or receptor agonists, any kinase or cell signaling assay may be employed. For kinase activity, assays that assess adenosine diphosphate (ADP) formation or the conversion of specific substrates may be used. For example, kinase activity may be monitored with commercially available kinase activity assessment kits such as but not limited to the ADP-Glo™ Kinase Assay and the Universal Kinase Activity Kit and Phospho-Kinase Antibody Array.

The LIN-28 target kinase may be part of a network of signaling pathways. The candidate agent may modulate the activity of the LIN-28 target kinase through other molecules in the network. The LIN-28 target kinase substrates, which may be used to design kinase activity assays, may also be identified as part of the signaling transduction network. Maps of kinase signaling pathways are known in the art. See e.g. upload.wikimedia.org/wikipedia/commons/f/fb/Signal_transduction_pathways.png; www.nature.com/nrc/journal/v10/n12/fig_tab/nrc2967 F4.html; physrev.physiology.org/content/91/1/177, which are incorporated by reference in their entireties.

In some embodiments, the LIN-28 target is a methyltransferase. For example, the LIN-28 target may be lysine methyltransferase (e.g. KMT2E and SETD1A) or N-terminal methyltransferase (e.g. METTL11B), which correspond to human orthologs of LIN-28 targets identified in Table 1.

The candidate agent may be an agonist or inhibitor of a methyltransferase or pathway comprising the same. In some embodiments, candidate agents may include molecules known to interact with one or more lysine methyltransferases or N-terminal methyltransferases, or which impact a cell pathway comprising the same. The basic reactions mediated by lysine methyltransferase and N-terminal methyltransferase enzymes are known and signal transductions described in en.wikipedia.org/wiki/Methyltransferase are hereby incorporated by reference.

In some embodiments, the candidate agents are tested for modulation of the activity of the target in a methyltransferase assay. When testing the library of candidate agents, such as a library of methyltransferase inhibitors or inhibitors or activators of the associated pathway, assays for the expression and/or activities of the LIN-28 target methyltransferase may be used. For example, the activity of lysine methyltransferase such as KMT2E and SETD1A may be monitored with assays such as but not limited to: detection of methylation with mass spectrometry using unlabeled S-Adenosyl methionine (AdoMet); immuno-assays using antibodies against different methylation states of lysines (e.g., in the detection of histone methylation in fixed chromatin); and detection of reaction turnover or detection of reaction products (e.g., the methyl donor product S-adenosy-L-homocysteine is enzymatically hydrolyzed to homocysteine and adenosine during the reaction, and the homocysteine concentration is then determined). The KMT2E activities may also be monitored with a continuous peptide methylation assay as disclosed in Rathert P. et al. 2007, which is hereby incorporated by reference. The activity of the N-terminal methyltransferase such as METTL11B may be monitored, for example, with the methylation assay described in Webb K J et al. 2010, which is hereby incorporated by reference.

In some embodiments, the LIN-28 target is a phosphatase. For example, the LIN-28 target may be PTPRN, PTPN23, PPP2R3C, PPP2CB, PPP1R37, PPP1R16A, or PDXP, which correspond to human orthologs of LIN-28 targets identified in Table 1.

The candidate agent may be an activator or inhibitor of a phosphatase or pathway comprising the same. In some embodiments, the candidate agents may include molecules that interact with or affect activity of phosphatases, including one or more of PTPRN, PTPN23, PPP2R3C, PPP2CB, PPP1R37, PPPIR16A, or PDXP. The basic reactions mediated by phosphatases are known and networks and signaling pathways are disclosed in FEBS Journal, Special Issue: Protein Phosphatases: From Molecules to Networks, 280(2), 2013, which are hereby incorporated by reference.

In some embodiments, the candidate agents are tested for modulation of the activity of the target in a phosphatase molecular or cellular assay. When testing the library of candidate agents, such as a library of phosphatase inhibitors or candidate molecules impacting a pathway comprising the same, assays for the activity of the LIN-28 target phosphatase (or cellular pathway comprising the same) may be employed. For example, the activity of phosphatase may be monitored with protein dephosphorylation assays such as but not limited to the phosphatase assays described in McAvoy T. et al. 2011, which is hereby incorporated by reference, and the commercially available ProFlouro® Ser/Thr Phosphatase Assay.

In some embodiments, the LIN-28 target is a transcription factor or helicase. For example, the LIN-28 target may be PAX6, DDX1, SMAD7, ARID1A, SMAD4, POU2F1, WRN, CHD9, ARID2, ARID3C, BCL11A or JARID2, which correspond to human orthologs of LIN-28 targets identified in Table 1.

The candidate agent may be an agonist or inhibitor of a transcription factor or helicase. In some embodiments, the library of candidate agents may include molecules that interact with transcription factors or helicases, including but not limited to PAX6, DDX1, SMAD7, ARID1A, SMAD4, POU2F1, WRN, CHD9, ARID2, ARID3C, BCL11a or JARID2. The basic reactions mediated by transcription factors and helicases are known.

In some embodiments, the candidate agents are tested for modulation of the activity of the target in a transcription, polynucleotide-binding, helicase, gene-expression, or cell proliferation assay. For example, transcription and polynucleotide-binding activities may be monitored with the electrophoretic mobility shift assay or high throughput assays such as but not limited to immobilized transcription factor arrays, microsphere assay for transcription, chromatin immunoprecipitation assays, oligonucleotide arrays and ELISA based transcription factors assays, and the assay described in Perkel J M 2006. Alternatively, or in addition, candidate agents can be evaluated in gene expression assays testing for activation or inhibition of the transcription factor. Such assays may use any of the known reporter systems, including but not limited to fluorescent or luminescent reporter genes. Helicase activities may be monitored with a number of assays which include but are not be limited to: strand displacement assays, rapid quench-flow assays, fluorescence-based assays, filtration assay, scintillation proximity assay, time resolved fluorescence resonance energy transfer assay, flashplate technology assay, homogeneous time-resolved fluorescence quenching assay and electrochemiluminescence-based helicase assay and the assays described in Tuteja N. et al. 2004, which is incorporated by reference. Helicase activities may also be evaluated according to cellular gene expression or cell proliferation assays.

In some embodiments, the LIN-28 target is a ribosyltransferase. For example, the LIN-28 target may be SIRT4, which corresponds to human orthologs of LIN-28 targets identified in Table 1.

The candidate agent may be an agonist or inhibitor of a ribosyltransferase. In some embodiments, the candidate agents may include molecules that interact with ribosyltransferases (such as but not limited to SIRT4) or the signaling transduction pathway molecules associated with ribosyltransferases. The basic reactions mediated by ribosyltransferases, such as deactylation, are known.

In some embodiments, the candidate agents are tested for modulation of the activity of the target in a ribosyltransferase assay or cellular assay based on a pathway involving the ribosyltransferase. For example, activities may be monitored with ADP-ribosylation assays measuring the transfer of adenosine diphosphate ribose (ADP-ribose) from nicotinamide adenine dinucleotide (NAD) onto specific target proteins. The activity of SIRT4 may be measured with the assays described in Du J. et al. 2009, which is hereby incorporated by reference.

In some embodiments, the LIN-28 target is a DNA or RNA polymerase. For example, the LIN-28 target may be POLD2 or POLR2A, which correspond to human orthologs of LIN-28 targets identified in Table 1.

The candidate agent may be an activator or inhibitor of a DNA or RNA polymerase. In some embodiments, the library of agents may include molecules that interact with DNA or RNA polymerases and/or impact polymerase activity (such as but not limited to POLD2 or POLR2A). The basic reactions mediated by DNA or RNA polymerases known.

In some embodiments, the candidate agents are tested for modulation of the activity of the target in a DNA or RNA polymerase assay, or cell proliferation assay. For example, the polymerase activity may be assessed with methods that measure incorporation of radiolabeled nucleotides, fluorescence generated by DNA polymerase-mediated release of single-stranded binding protein, or binding of PicoGreen™ to double-stranded DNA. The polymerase activity may also be monitored with assays and method described in Zweitzig et al. 2012, which is incorporated by reference.

In some embodiments, the LIN-28 target is an E3 ubiquitin ligase. For example, the LIN-28 target may be SKP1 or ARIH2, which correspond to human orthologs of LIN-28 targets identified in Table 1.

The candidate agent may be an agonist or inhibitor of a ubiquitin ligase. In some embodiments, the candidate agents may include molecules that interact with ubiquitin ligases (such as, but not limited to, SKP1 or ARIH2) or the signaling transduction pathway molecules associated with ubiquitin ligases. The basic reactions mediated by ubiquitin ligases are known.

In some embodiments, the candidate agents are tested for modulation of the activity of the target in a ubiquitin ligase assay. For example, the activity of a ubiquitin ligase may be measured with assays that assess ligated protein levels and/or high throughput assays such as the assay described in Davydov I V et al. 2004, which is hereby incorporated by reference. In one embodiment, the candidate agents may be tested for modulation of the activity of the LIN-28 target in an E3 ubiquitin ligase assay such as but not limited to the Abcam® E3 Ligase Auto-Ubiquitylation Assay.

In some embodiments, the LIN-28 target is in the mTOR signaling pathway. For example, the LIN-28 target may be RPTOR, which corresponds to a human ortholog of a LIN-28 target identified in Table 1.

The candidate agent may activate or inhibit the mTOR signaling pathway, which is known in the art and described in Singh S S et al. 2015, which is hereby incorporated by reference. In some embodiments, the candidate agents include molecules that interact with RPTOR or other factors in the mTOR signaling pathway, or which modulate the mTOR signaling pathway.

In some embodiments, the candidate agents are tested for modulation of the activity of the mTOR pathway. For example, the mTOR pathway activity may be measured with the commercially available assays such as but not limited to the K-LISA™ mTOR Activity Assay, Phospho-mTOR Cellular Assay (Cisbio Inc.), mTOR (pSer2448) ELISA Assay (Abcam Inc.) and the assays described in Huang 2012, which is hereby incorporated by reference.

In some embodiments, the LIN-28 target is a protease. For example, the LIN-28 target may be ADAMTS4 or ADAM18, which corresponds to a human ortholog of a LIN-28 target identified in Table 1.

The candidate agent may be an agonist or inhibitor of a protease. In some embodiments, the candidate agents may include molecules known to that interact with proteases (such as ADAMTS4, ADAM18, or others) or the signaling transduction pathway molecules associated with these proteases. The basic reactions mediated by proteases are known.

In some embodiments, the candidate agents are tested for modulation of the activity of the target in a protease assay. For example, the activity of proteases may be measured with universal protease activity assays using casein as a substrate (from Sigma-Aldrich®), Proteasome-Glo™ Assay (Promega Inc.), or the Abcam® protease activity assay.

In some embodiments, the LIN-28 target is a phosphodiesterase. For example, the LIN-28 target may be PDE2, which corresponds to a human ortholog of a LIN-28 target identified in Table 1.

The candidate agent may be an activator or inhibitor of a phosphodiesterase, or cell pathway comprising the same. In some embodiments, the library of candidate agents may include molecules known to interact or affect the activity of phosphodiesterases (such as PDE2 or others) or the signaling transduction pathway molecules associated with phosphodiesterases. The basic reactions mediated by phosphodiesterases are known.

In some embodiments, the candidate agents are tested for modulation of the activity of the target in a phosphodiesterase assay. For example, the activity of phosphodiesterases may be measured with PDELight™ HTS cAMP Phosphodiesterase Assay (from Lonza®), PDE-Glo™ phosphodiesterase Assay (Promega Inc.), or the Abcam® PDE activity assay.

In some embodiments, the candidate agent is an antisense polynucleotide or siRNA that targets an mRNA of a gene in Table 1. In some embodiments, the candidate agent is a small RNA (such as a microRNA or mimic thereof) that increased or decreases the expression of a gene in Table 1.

In some embodiments, the candidate agent will be intended to bind to (and often inhibit) its intended target. Thus, the candidate agent may be an antibody or antigen-binding fragment thereof, or other binding agent such as a peptide, aptamer, adnectin, polysaccharide, or biological ligand. The various formats for target binding include a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317. Exemplary targeting agents include antigen-binding antibody fragments, such as but not limited to F(ab′)2 or Fab, a single chain antibody, a bi-specific antibody, or a single domain antibody.

In still other embodiments, the candidate agent will be, or will mimic, a polynucleotide. For example, the candidate agent may be a polynucleotide of from about 8 to about 30 nucleotides in length, and may include one or more chemical modifications making the polynucleotide compatible with therapeutic applications. Desirable chemistries in these embodiments can include locked nucleic acid (LNAs) and bridged “bicyclic” nucleotides. LNAs are described, for example, in U.S. Pat. Nos. 6,268,490, 6,316,198, 6,403,566, 6,770,748, 6,998,484, 6,670,461, and 7,034,133, all of which are hereby incorporated by reference in their entireties. LNAs in some embodiments contain an extra bridge between the 2′ and 4′ carbons of the ribose sugar moiety resulting in a “locked” conformation, and/or bicyclic structure. In some embodiments, at least 25% of the nucleotides are LNAs.

Polynucleotide agents may further comprise a 2′ modification with respect to a 2′ hydroxyl. For example, the 2′ modification may be 2′ deoxy. Incorporation of 2′-modified nucleotides in antisense oligonucleotides may increase both resistance of the oligonucleotides to nucleases and their thermal stability with complementary RNA. Various modifications at the 2′ positions may be independently selected from those that provide increased nuclease sensitivity, without compromising molecular interactions with the RNA target or cellular machinery. In some embodiments, the 2′ modification may be independently selected from O-alkyl (e.g., O-methyl), halo, and deoxy (H).

In certain embodiments, the oligonucleotide further comprises at least one terminal modification or “cap”. The cap may be a 5′ and/or a 3′-cap structure. The terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the oligonucleotide to exonucleases without compromising molecular interactions with the RNA target or cellular machinery. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety.

The oligonucleotide may contain one or more phosphorothioate linkages. Phosphorothioate linkages have been used to render oligonucleotides more resistant to nuclease cleavage. For example, the polynucleotide may be partially phosphorothioate-linked, for example, phosphorothioate linkages may alternate with phophodiester linkages. In certain embodiments, however, the oligonucleotide is fully phosphorothioate-linked.

According to one aspect of the invention, the modulation of the expression and/or activity of the LIN-28 target may be confirmed in an animal model. The animal models may include but are not limited to tumor or cancer models in rodents such as mice and rats, and include assays based on inhibiting or slowing tumor growth or inhibiting metastasis, and/or other measurable cancer-related phenotypes.

After identifying the agent that modulates expression and/or activity of the LIN-28 target, the selected agent may be formulated as a pharmaceutically-acceptable composition, which may be used to treat LIN-28 expressing cancer. The pharmaceutical composition may be formulated into liquid or solid dosage forms and administered systemically or locally. The pharmaceutical composition may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes for which the agent can be formulated include oral, buccal, by inhalation spray, sublingual, rectal, transdermal, vaginal, transmucosal, nasal or intestinal administration; parenteral delivery, including intramuscular, subcutaneous, intratumoral, intramedullary injections, as well as intrathecal, direct intraventricular, intravenous, intra-articular, intra-sternal, intra-synovial, intra-hepatic, intralesional, intracranial, intraperitoneal, intranasal, or intraocular injections.

In a related aspect, the present invention provides a method of treating LIN-28 expressing cancer, by administering to a patient in need thereof, a pharmaceutical composition made according to the present disclosure. The patient is generally a cancer patient having a LIN-28-expressing or over-expressing cancer. For example, the tumor cells are over-expressing LIN-28, as compared to non-tumor differentiated cells. Expression of LIN-28, or expression level of one or more LIN-28 targets, may be evaluated or confirmed in a tumor or tissue biopsy, or cell culture derived therefore, of the subject's cancer. Agents prepared according to the present disclosure, are particularly suitable for therapy, for patients that test positive for LIN-28 expression in tumor biopsies, or test positive for the expression of activity of one or more LIN-28 targets.

Thus, the invention provides companion diagnostic assays for cancer treatment. Specifically, the invention allows cancer biopsies to be tested for LIN-28 or LIN-28 target expression or activity, so that candidate agents (including those identified by the methods described herein) can be appropriately selected for treatment on a personalized basis.

The LIN-28 expressing cancer may be any cancer, or any malignant tumor or neoplasm with a cancerous cell population expressing LIN-28. In some embodiments, the cancer is colon cancer, breast cancer, lung cancer, liver cancer, pediatric cancer (e.g. neuroblastoma, wilms tumors) and cervical cancer, which have been identified to include cell populations to over-produce LIN-28. See Viswanathan et al. 2009, which is incorporated by reference in its entirety.

Molecular assays for the expression and/or activity of LIN-28 or the LIN-28 target include immunochemical assays, nucleic acid hydridization assays, RT-PCR, and DNA sequencing, among others.

### EXAMPLES

### Materials and Methods

### HITS-CLIP:

High-throughput sequencing (HITS) of RNA isolated by crosslinking immunoprecipitation (CLIP) experiments were performed as follows. C. elegans transgenic strains carrying a single copy of a modified lin-28 gene, encoding a fusion GFP, flag, HAHA at the C-terminus, were generated by bombardment. The expression of the transgene at the proper time and place was verified by RT-PCR, western blot and by its ability to fully rescue the phenotype of the lin-28(n719) mutant strain. Liquid cultures of staged, fed L1 larvae (containing about five million animals) were harvested by centrifugation, washed in M9 solution, and treated with UV in a Stratalinker (3.6 mJ/cm2). Subsequently, worms were lysed with zirconia beads by three 20 seconds cycles in a MP Fastprep 24 in buffer A (20 mM Hepes pH 7.4, 150 mM NaCl, 0.1% SDS, 0.5% deoxycholate, 0.5% NP40, 20 mM EDTA and 20 mM EGTA). The lysate was cleared by ultracentrifugation (100,000×g, 30 minutes). Subsequent steps were performed as described previously, with few modifications (Jensen and Darnell, 2008; Ule, et al., 2005). LIN-28/RNA complexes were purified with a commercial antibody anti-HA (HA-7, Sigma H3663) conjugated with Dynabeads (Life Technologies 112-01D). During the subsequent washing steps, the complexes were treated with an optimized amount of micrococcal nuclease to achieve an average RNA size of about seventy nucleotides, as estimated by gel electrophoresis. A 5′ end adapter (5′-/5AmMC6/AGGGAGGACGAUGCGG-3′, SEQ TD NO: 1) was ligated overnight. Following SDS-PAGE purification and proteinase K treatment, a 3′ end adapter (5′-P-GUGUCAGUCACUUCCAGCGG-Pmn, SEQ ID NO: 2) was ligated, and Reverse Transcription/PCR was performed (forward primer: 5′-AATGATACGGCGACCACCGACTATGGATACTTAGTCAGGGAGGACGATGC GG-3′ (SEQ ID NO: 3), reverse primer: 5′-CAAGCAGAAGACGGCATACGACCGCTGGAAGTGACTGACAC-3′ (SEQ ID NO: 4)). Libraries thus prepared were sequenced in an Illumina HighSeq 2000 machine using primer 5′-CTATGGATACTTAGTCAGGGAGGACGATGCGG-3′ (SEQ ID NO: 5). RNA-seq libraries were performed from total RNA purified from L1 larvae reared the same way, following oligo(dT) selection, according to the standard Illumina protocol.

### RNA-CoIP, RT-qPCR:

RNA co-IP, followed by qPCR where performed as follows: C. elegans larvae were harvested, UV-treated and lysed as described above. Following clearing by ultracentrifugation and pre-incubation with beads conjugated with mouse IgG, protein RNA-complexes were purified using anti-HA antibodies (HA-7, Sigma H3663) conjugated with Dynabeads (Life Technologies 112-01D). After overnight incubation at 4° C., complexes were washed three times with buffer A (see above), three times with buffer B (20 mM Hepes pH 7.4, 300 mM NaCl, 0.1% SDS, 0.5% deoxycholate, 0.5% NP40, 20 mM EDTA and 20 mM EGTA) and once with buffer E (100 mM Tris-HCl, pH 7.4, 50 mM NaCl, 10 mM EDTA). During these washes, the complexes were treated with DNAse (Turbo DNAse, Ambion). Finally, RNA was eluted by treatment with proteinase K followed by two phenol-chloroform extractions and precipitation. Reverse transcription was performed using random hexamers and Superscript III (Life Technologies). Mature let-7 was detected using a Taqman Assay (Life technologies). Quantitative PCR was conducted in a Roche Lightcycler LC480.

### Protein-RNA In Vitro Cross-Linking:

RNA was transcribed in vitro using T7 RNA polymerase and a 134 base pairs DNA template corresponding to the LIN-28 binding site identified by CLIP (WT), a version of the same sequence where the four GGAG sequences were mutated to CTCC (MUT), a scrambled sequence with the same nucleotide composition as WT (C-), and the pre-let-7 distal loop (pre-/et-7). The transcription mix contained cold GTP and P32-labeled GTP (in a 2.8:1 molar ratio). In vitro transcribed RNA was gel-purified before the assay. C. elegans larvae protein extract was prepared as described above, using a different lysis buffer (20 mM Hepes, pH 7.4, 150 mM NaCl, 0.2% NP40, 3 mM MgCl₂, 1 mM DTT). Equal counts of RNA (roughly corresponding to 20 (moles) were heated at 65° C. for 5 minutes, then incubated with C. elegans larvae protein extract (300 μg of total protein) for 10 minutes at 30° C. in 100 in the presence or absence of cold competitor RNA. At the end of the incubation, the reaction mix was crosslinked for 15 minutes on ice in a 48-wells plate in a Stratalinker. After immune-purification, the protein-RNA complexes were washed and treated with micrococcal nuclease (NEB, diluted 1:100) for 10 minutes at 37° C. After further washes, the protein-complexes were eluted in SDS-PAGE sample buffer at 80° C. for 10 minutes, resolved on a 4-12% Bis-Tris gel (Biorad) and transferred to a nitrocellulose membrane. The membrane was exposed to a phosphoimager and to film.

Data processing

Reads from both CLIP and RNAseq experiments were mapped to the C. elegans genome version WS190/cc6 using Novoalign. The program can remove adapters at the read ends and allow identification of substitutions and small indels in the reads. To exclude ambiguous regions, only reads that mapped to exon regions and miRNA regions were considered. Since most of the genes in Refseq database in UCSC genome browser lack UTR annotation, 200 bp at 5′ end and 750 bp at 3′ end were extended based on the known average UTR length (95% quantile of UTR length, 5′UTR: ˜200 bp, 3′UTR: ˜450 bp) in Wormbase and the mapped tag density around coding regions. Then the overlapping exon regions were concatenated to generate the target exon regions for subsequent analysis. For miRNAs, pre-miRNA coordinate information was downloaded from MirBase (version 13.0), and then extended 1000 bp up and downstream to generate putative pri-miRNAs. To avoid confusion coming from reads of exon regions, the extended regions overlapped with exons defined above were cut to the position right after the exons, and the miRNAs were discarded if pre-miRNA regions overlapped with exons. Reads that mapped to the exons or miRNAs were extracted and summarized for 150 bp windows. Since our CLIP-seq data was generated from strand-specific sequencing, it was summarized for each of the forward and reverse strands separately. On the other hand, RNAseq data was generated from two-stranded sequencing, so the two strands were combined to give the final counts for each window.

### CIMS (Crosslinking Induced Mutation Site)Analysis

To accurately obtain potential binding sites with crosslinking induced mutations, the mutation patterns induced by cross-linking in CLIP-seq were first examined. In order to determine the subtype of the mutations representing cross-linking sites, three types of mutations were summarized and analyzed—substitution, deletion and insertion. Mutations were clustered if they were mapped at the same position. For mutations longer than 1 bp, only the first base was considered. To distinguish CIMS from sequencing errors, the mutation positions were ranked with a Binomial test (equation 1) from the hypothesis testing whether the proportion of reads with mutation in the position is significantly higher than that in the whole genome. The p-values were adjusted for multiple testing using Benjamini-Hochberg (BH) method (Benjamini and Hochberg 1995).

$$pvalue ( a \vert y , p ) = {\sum }_{x \geq a} ( \begin{matrix}y \\ x\end{matrix} ) {{p}^{x} ( 1 - p )}^{y - x} \\ where \\ p = \frac{\# of mutation type}{\# of reads * read length} \qquad ( 1 )$$

where a is the number of mutations at the position and y is the total number of reads mapped to that position. Ambiguous mutations were filtered using the following criteria. First, sequencing technology usually introduces errors on repeated tandem sequences (e.g. region containing a sequence of same nucleotides, such as TTTT), so the surrounding regions of mutation positions were extracted and those on nucleotide tandem sequences with at least 5 repeats were excluded. Second, to avoid PCR amplification biases, mutation clusters containing at least three uniquely mapped mutations were required (e.g. from three unique reads).

After filtering, the top 500 mutation positions ranked with BH adjusted p-values (<=0.05 required) in each mutation type were extended 15 bp up and downstream, and then the sequences were extracted from UCSC genome browser and subjected to the MEME algorithm to identify motifs (Bailey et al. 2009). To see the enrichment levels of motifs, the motif identified from deletion clusters in all mutation positions using the FIMO algorithm (Grant et al. 2011) were searched. The resolution of CIMS analysis on binding site identification was obtained by considering motif distance from positions of deletion clusters.

### Peak Analysis

A combined parametric model with dynamic Poisson and negative binomial regression was used to obtain the putative binding sites from tag counts. RNAseq data was used as a matching control for CLIPseq.

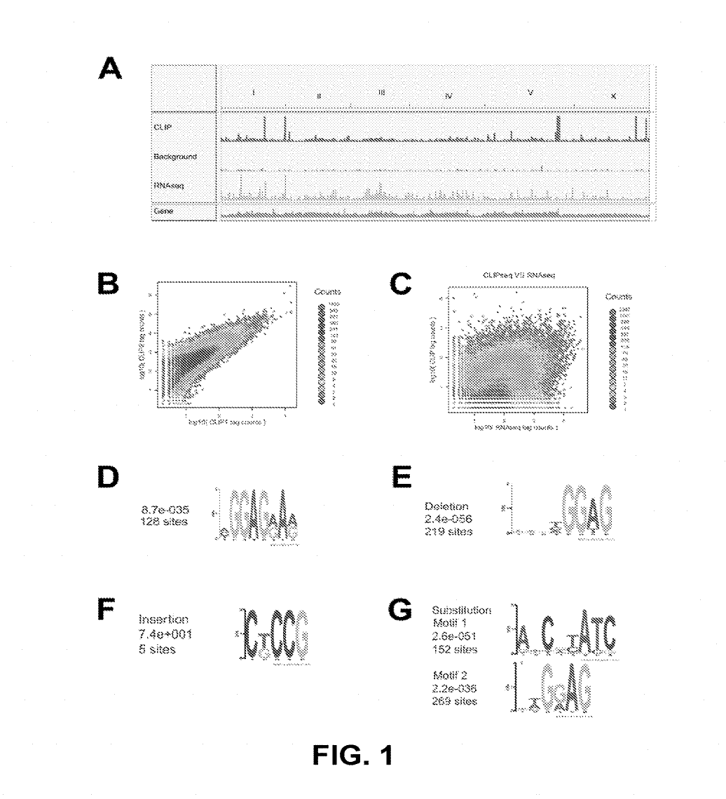

Top 500 peak windows were extended 100 bp up- and downstream and then subjected to MEME to search for motifs. The motif with the best E-value was selected as the motif identified by peak analysis. Top 2000 peak windows were selected for binding features analysis, such as binding distribution on transcripts, resolution of binding sites and GO analysis. The resolution of binding site identification by peak analysis was obtained by considering the distance of window center to high confident mutations (top mutations from deletions and substitutions) defined binding sites. Only real peaks that emerged in each independent experiment were considered. Despite the difference in sequencing depth, the identified binding sites and read distribution pattern are very similar in two repeats, as shown in FIG. 1, panel B. Almost all the peaks found in the less deep dataset (referred to as CLIP1) are also present in the deeper dataset (CLIP2) as well. The main difference is that the CLIP2 covers wider genomic regions, but most of those regions are covered by fewer than 10 tags, which suggests they may represent background.

Binding Site Identification in microRNA Regions

Since RNAseq is specifically designed to study mRNAs, it is not suitable to be used as the matching control for microRNA regions. Thus, one-sample analysis without control was applied on microRNA regions. To consider the possible overdispersion of the CLIPseq data, a negative binomial model (equation 1) was used to identify the binding sites in microRNA regions. The parameters were estimated using maximum likelihood estimation method. P-values were adjusted with Benjamini-Hochberg (BH) method.

$$p ( x \vert μ , α ) = \frac{Γ ( x + {α}^{1} )}{x ! Γ ( {α}^{- 1} )} {( \frac{μ}{μ + {α}^{- 1}} )}^{x} {( \frac{{α}^{1}}{μ + {α}^{- 1}} )}^{1 / α} \qquad ( 1 )$$

### Go Analysis:

The Refseq IDs of the genes corresponding to the top 1,500 binding sites (suppl. Table 2) were analyzed with the Functional Annotation Clustering Tool of the David website (david.abcc.ncifcrfgov) with the following parameters: Classification Stringency: Highest; Similarity Term Overlap: 3; Similarity Threshold: 1; Initial and Fin al Group Membership: 3; Multiple Linkage Threshold: 0.50; Enrichment Threshold EASE: 1.0; Display: Benjamini

### Mapping of HITS-CLIP Libraries

Living late L1 stage animals were exposed to UV light to cross-link proteins and RNAs in situ. In vivo cross-linked RNA was co-purified with a rescuing LIN-28 fused to HA tag and characterized by high throughput sequencing. As a control for background, samples were isolated and prepared in an identical manner from a strain lacking the HA tag.

6,727,518 reads were obtained from CLIPseq 1 and 206,665,887 reads from a second biological replicate, CLIPseq 2. The reads from the CLIP experiments were mapped to the C. elegans genome version WS190/ce6 by Novoalign (novocraft.com). About 75% of reads generated by HITS-CLIP (high-throughput sequencing of RNA isolated by crosslinking immunoprecipitation) (5,087,544 for CLIPseq 1 and 156,886,622 for CLIPseq2) could be mapped to the C. elegans genome, yielding a complete snapshot of LIN-28/transcriptome interactions at the L1 stage (FIG. 1, panel A). The read depth distribution by 150 bp windows of exon regions between experimental trials shows a high level of reproducibility with a correlation coefficient of 0.803 (FIG. 1, panel B). The relatively poor correlation (0.455) between read depth in CLIP samples and RNA abundance (RNAseq) reveals that CLIP captures specific protein-RNA interactions and is not overly affected by transcript abundance; however, a correlation level of 0.455 also indicates that RNAseq can be treated as a good matching control for exon regions (FIG. 1, panel C).

LIN-28 binding sites were identified by a novel CLIP data analysis pipeline that relies on both peak analysis and crosslinking induced mutation site (CIMS) analysis (Kishore et al. 2011) (Zhang and Darnell 2011). For peak analysis, a parametric model was devised based on combination of dynamic Poisson and negative binomial regression models to identify and quantify binding events. The CIMS analysis is made possible by the occurrence of mutations in the reverse transcription of RNA molecules that had been cross-linked to protein, likely due to residual peptides disrupting the fidelity of cDNA synthesis by Reverse Transcriptase (Zhang and Darnell 2011).

The CLIP data analysis revealed that LIN-28 binds an excess of two thousands mRNA sites in vivo. Within this dataset of candidate target sequences, the presence of shared enriched motifs was identified with the Multiple Expectation maximization for Motif Elicitation (MEME) algorithm (Bailey et al. 2009). In order to evaluate the consistency of motif identification between two analyses, MEME searches were conducted within the target sets obtained by peak analysis and CIMS separately.

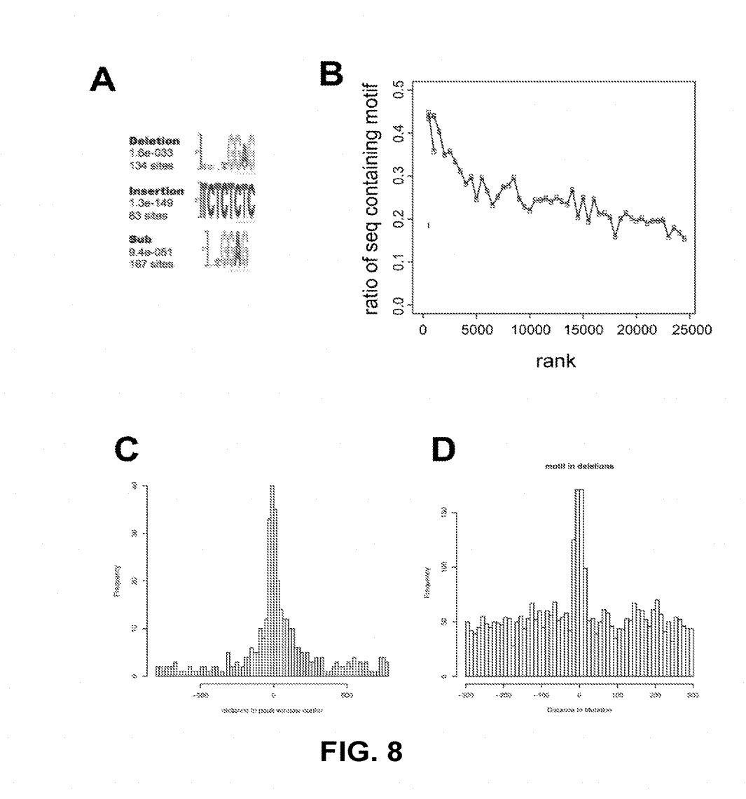

Within the peak analysis dataset, a top-scoring motif was identified with length 8 bp with score 8.7e-035 containing the GGAG quadruplet, similarly to the datasets generated in vertebrate cells (FIG. 1, panel D). The target set obtained by CIMS were evaluated separately to identify three types of mutations: deletions, insertions and substitutions. The sequence tags identified by deletions presented motifs similar to the ones predicted based on peak analysis, a 6 bp motif containing GGAG (FIG. 1, panel E). However, this pattern was not present in the sets generated on the basis of insertions (FIG. 1, panel F). Within the binding sites identified by substitutions, a GGAG-containing element was identified alongside a different motif (FIG. 1, panel G and FIG. 8, panel A). High motif enrichment in high confident deletions (˜900) and substitutions (˜top 2000) of CLIP1 also shows that these two types of mutations contain relatively high proportion of CIMS; however, lower ranked substitutions might be diluted by sequencing errors and SNPs in the sample (FIG. 8, panel B). Thus, deletion (BH<=0.05) appears to be the primary mutation type induced by cross-linking to proteins in the CLIP protocol, but substitution (BH<=0.05) also contains a proportion of crosslinking information. Furthermore, CIMS analysis contributes significantly to pinpoint accurate sites of protein-RNA interactions, as the average length of binding site sequences from peak analysis is around 300 nucleotides (FIG. 8, panel C), while it is about 40 nucleotides for CIMS (FIG. 8, panel D).

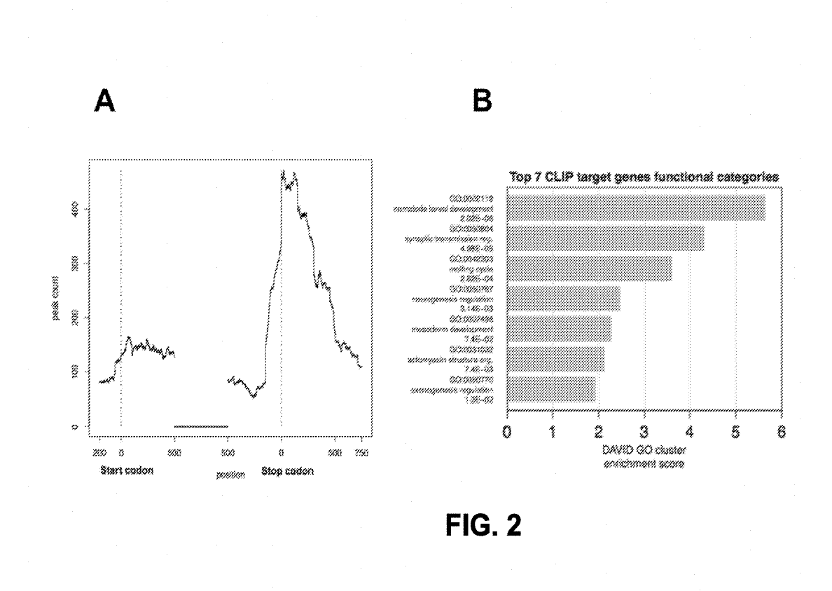

The binding sites distribution within transcripts shows a marked under-representation in the 5′ UTR (3.96%) compared to coding sequence (56.52%) and 3′ UTR (39.52%) (FIG. 2, panel A). Nonetheless, given that 3′UTRs are on average shorter than coding sequences, the highest enrichment of CLIP tags per sequence length is observed in the former. For each region type (5′UTR, CDS and 3′UTR), an enrichment score was calculated based on

$${EnrichScore}_{region} = \frac{\# of peaks in region}{length of region / \# of genes} ,$$

region:5′UTR, 3′UTR or CDS

The score for each region type is 5′UTR 0.700, CDS 1.495 and 3′UTR 1.864. Thus peaks are mostly enriched at 3′UTRs. Notably, the highest abundance of peaks within coding regions is also near their 3′ ends (FIG. 2, panel A).

Overall, the sole enrichment within the dataset of the GGAG motif, which has been extensively validated through mutational and structural studies in the context of Lin28 binding to let-7 terminal loop, indicates the validity of the bona fide target sequences identified by CLIP.

### LIN-28 Target Transcripts Identified

The analysis of the CLIP dataset identified an excess of 2000 in vivo LIN-28 binding sites. A search for over-represented terms in the Gene Ontology (GO) database showed a notable enrichment of biological process terms related to animal development (FIG. 2, panel B). Nematode larval development is the most highly enriched category, consistently with the well-established role of lin-28 as a regulator of post-embryonic animal development.

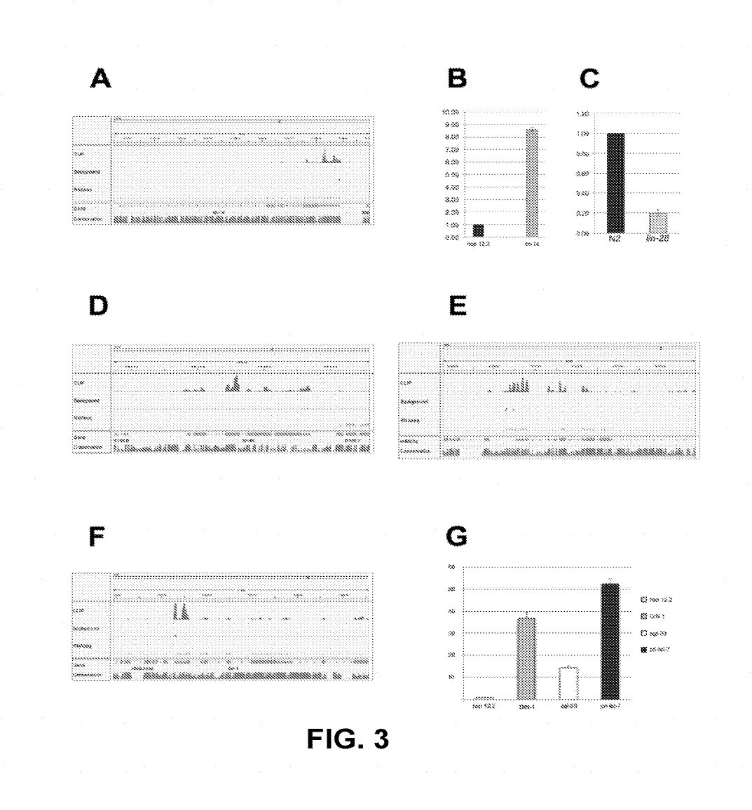

The data show that LIN-28 interacts with lin-14 mRNA, mostly within the 3′UTR (FIG. 3, panel A). This interaction was confirmed in independent experiments by RNA-co-immunoprecipitation (RIP) followed by qPCR (FIG. 3, panel B). Furthermore, the abundance of lin-14 mRNA is decreased in lin-28 mutants, suggesting that the previously documented positive effect of lin-28 on lin-14 protein levels is the result of an overall stabilizing effect on lin-14 mRNA (FIG. 3, panel C).

Forward genetic screens have identified lin-46 (ranked 604 in our list), another heterochronic gene, as a suppressor of lin-28 (Pepper et al. 2004). Our CLIP experiment documents extensive interactions of LIN-28 with lin-46 mRNA, both within the coding sequence and the 3′UTR, suggesting that at least part of the functional interaction is caused by a physical interaction between LIN-28 protein and mRNA (FIG. 3, panel D). LIN-28 also binds the mRNA of the developmental timing kinase gene kin-20, homolog of Drosophila clock gene doubletime (position 1013, FIG. 3, panel E). In addition, LIN-28 interacts with its own mRNA, suggesting that LIN-28 autoregulates its own expression. LIN-28 binds with the 3′UTR of din-1 mRNA, an interaction that was confirmed in separate RIP-qPCR experiments (FIG. 3, panels F, G).

These data show that LIN-28 interacts with a large population of transcripts during C. elegans development. While the functional implications of the vast majority of these interactions remain currently not understood and will be the subject of future investigation, a subset of the identified targets are known regulators of the timing of animal development, which, in the case of lin-14 and lin-46, were known to interact genetically with lin-28.

In addition, a subset of the LIN-28 interacting genes is shared with those interacting with the homologues of LIN-28, suggesting that these interactions have been conserved through evolution. Of the identified LIN-28 targets in C. elegans, 46% (537 out of 1168) have human orthologs. Of these, 97 (including LIN-28B) emerged as targets of LIN-28B in a previous study that characterized LIN-28 interactions with human transcriptome by PAR-CLIP. There is no clear enrichment in GO functional categories such as splicing factors or transmembrane protein products as reported by previous studies in mammalian cells.

The human orthologs of LIN-28 targets are identified based on the identifications of the C. elegans targets. The human LIN-28 targets are listed in Table 1.

Identification of LIN-28 Binding Site in C. elegans Pri-Let-7

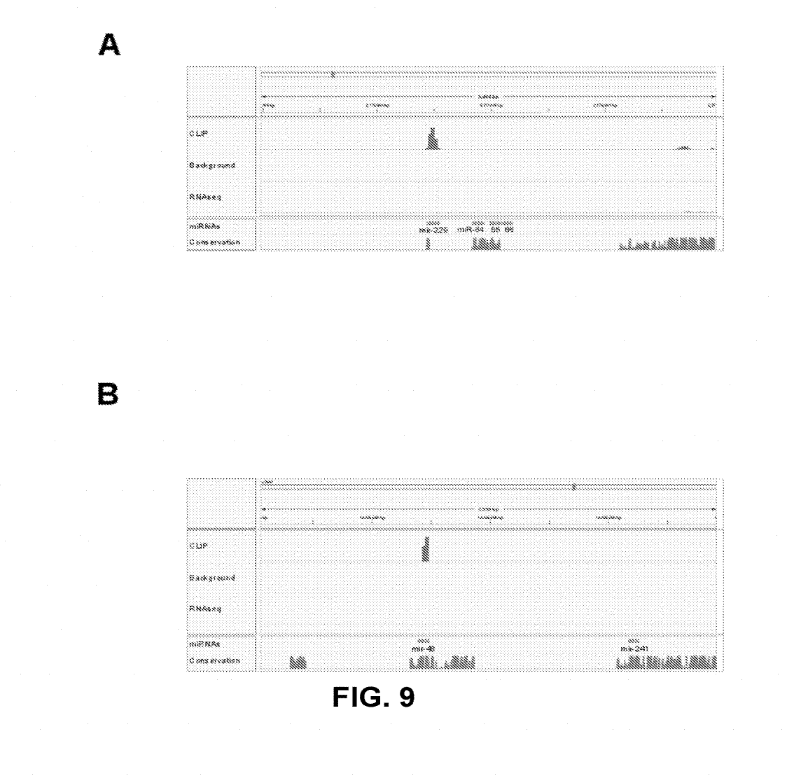

Interactions of C. elegans LIN-28 with genomic regions surrounding miRNAs were analyzed. Pri-let-7 is most significant candidate target, with the lowest adjusted p-value (3.87e-13) gained from a negative binomial test (see Methods). Additionally, two other pri-miRNAs appear to be bound by LIN-28 with high probability (FIG. 9, panels A and B). One of them, pre-miR-48, is a member of the let-7 family (FIG. 9, panel B). miR-48 and miR-241, another member of the let-7 family, are encoded less than 1,700 base pairs apart on the minus strand of chromosome V. Furthermore, LIN-28 binds pre-miR-229, a member of a group of four miRNAs clustered within less than a thousand base pairs (miR-64, miR-65, miR-66 and miR-229) on chromosome III (FIG. 9, panel A). The proximity of these miRNAs suggests that they might be transcribed as part of single primary transcripts encompassing the entire cluster; in such a scenario, LIN-28 could be involved in modulation of subsequent miR-229 or miR-48 maturation steps, decoupled from miR-64, 65, 66, or miR-241, respectively.

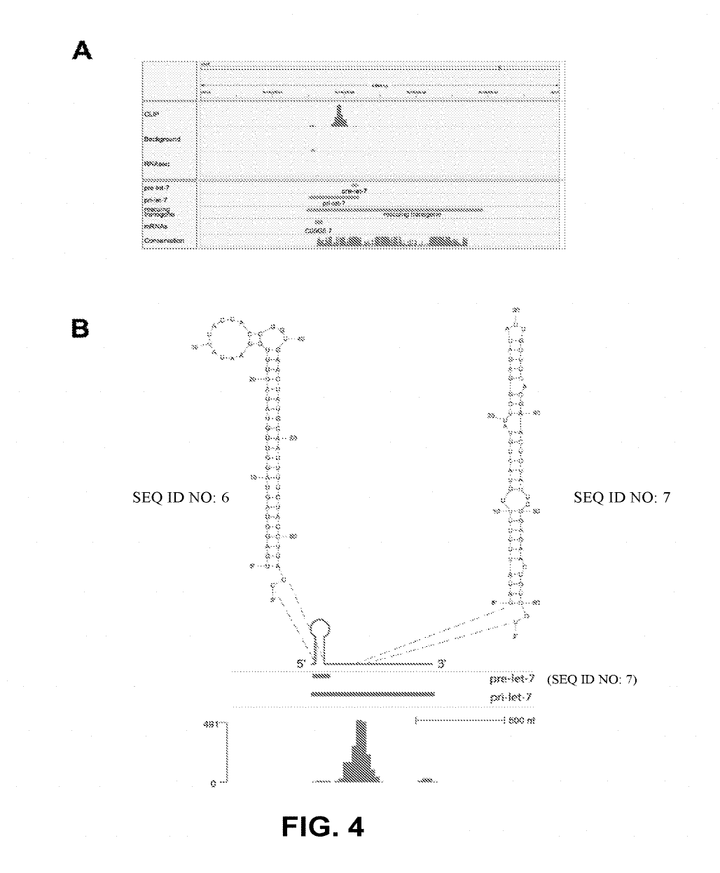

The terminal loop of C. elegans pre-/et-7 lacks a GGAG motif presenting a mystery as to how LIN-28 might bind to let-7. The HITS-CLIP experiments do not show an interaction with the terminal loop of let-7 (FIG. 4, panels A and B). Instead, LIN-28 appears to interact with a region of pri-/et-7 located 170 nucleotides downstream from the predicted 3′ end of pre-let-7 (FIG. 4, panels A and B; SEQ ID NO: 6). This novel LIN-28 binding site (LBS) contains two GGAG motifs within a region that can be folded to form a weak hairpin structure (SEQ ID NO: 7; predicted folding free energy: −11.70 kcal/mol, FIG. 4, panel B). Two additional GGAG motifs were found within thirty nucleotides of both ends of the LBS.

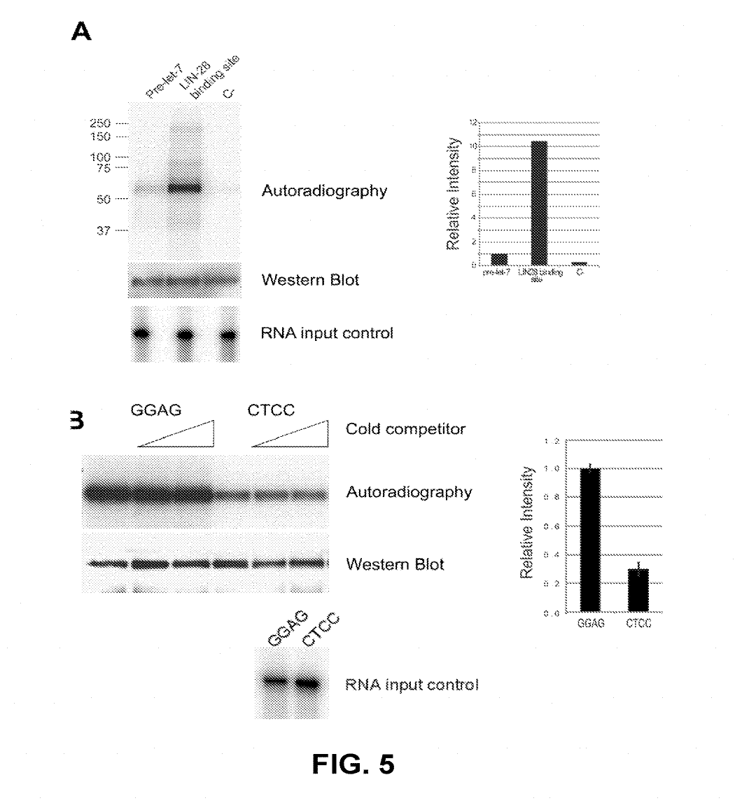

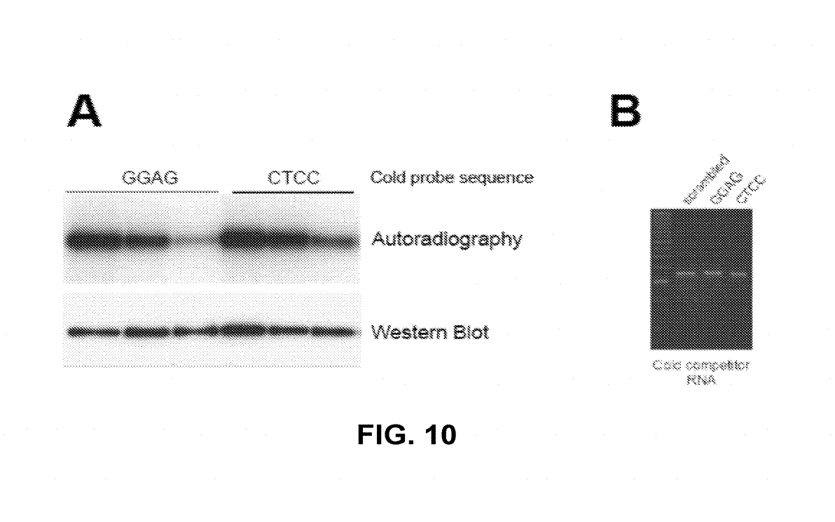

The binding of LIN-28 to the LBS was studied using an in vitro UV-crosslinking assay with radiolabeled RNA (see Methods for details). This assay revealed a markedly stronger interaction between LIN-28 and LBS RNA than an RNA of the same length corresponding to the pre-let-7 stem-loop structure (FIG. 5, panel A). A mutation of the GGAG motifs to CTCC within LBS drastically decreased the binding (FIG. 5, panel B). The addition of an unlabeled competitor RNA (with same base composition but scrambled sequence as the ‘GGAG’ probe) to the binding reaction does not affect the binding to LIN-28 of the GGAG nor CTCC mutant probes, demonstrating that both interactions are sequence-specific (FIG. 5, panel B). Incomplete reduction of binding was observed with mutation of GGAG repeats, as well as the ability of a CTCC cold competitor to affect binding, albeit with lower efficiency than the GGAG competitor (FIG. 10).

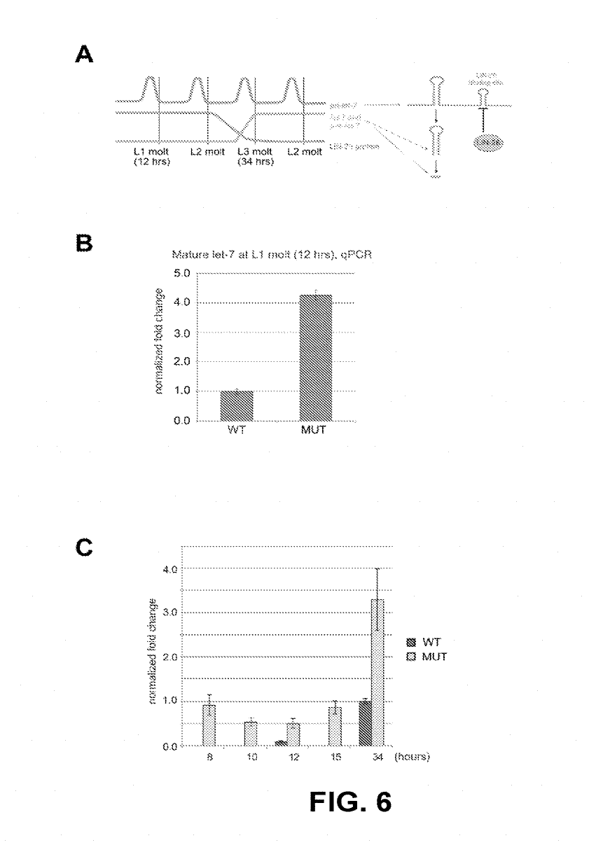

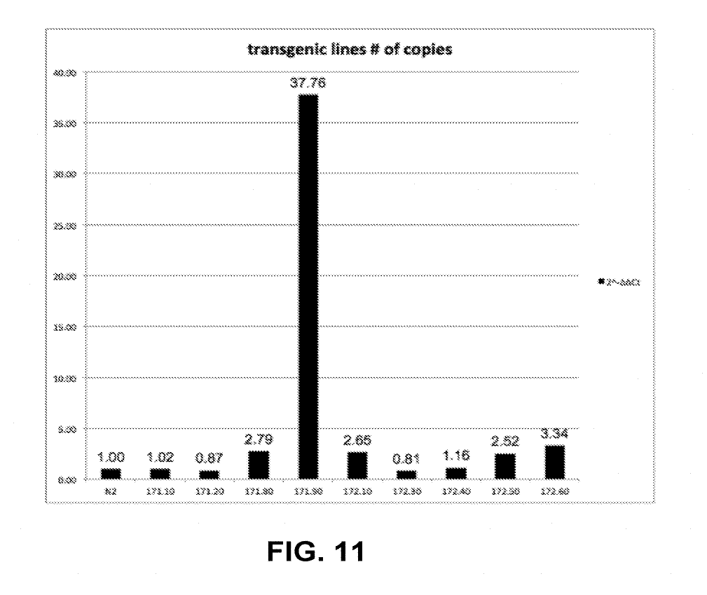

C. elegans transgenic lines carrying low-copy insertion of either a construct containing all the information for proper let-7 expression (2.5 kb let-7 rescuing fragment, Reinhart et al., 2000) were generated, or a version of the same construct in which the LBS was deleted (FIG. 11). Consistent with a role of LBS in mediating repression of maturation, its deletion resulted in a four-folds increase of the levels of mature let-7 at the time of L1 molt (FIG. 6, panel B). Furthermore, assaying for mature let-7 by qPCR at 2 hour intervals around the time of L1 molt showed that animals carrying the transgene lacking LBS produced an amount of mature let-7 similar to the amount detected in wild type transgenes at the normal time of mature let-7 appearance (34 hours, or L3 molt), while mature let-7 was virtually undetectable in wild type transgenes around the time of L1 molt (8, 10, 12, 15 hours) (FIG. 6, panel C). There was three-fold increase in the amount of mature let-7 at the L3 molt time point in the mutated transgene compared to the wild type, despite the same number of copies of transgene integrated in the genome as detected by qPCR. Upon elimination of LIN-28 by RNAi, there was a more marked derepression of let-7 maturation in animals carrying the WT let-7 transgene than in those expressing the pri-let-7 form mutated in the LBS (7.45 fold vs. 2.74 fold, p=3.75×10⁻⁴, student t test). These data identify a novel LIN-28 binding site in pri-let-7 in nematodes.

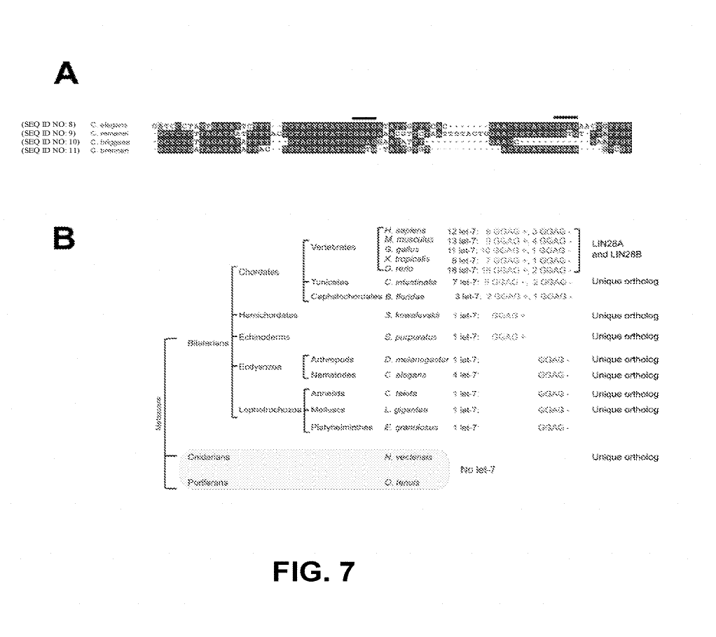

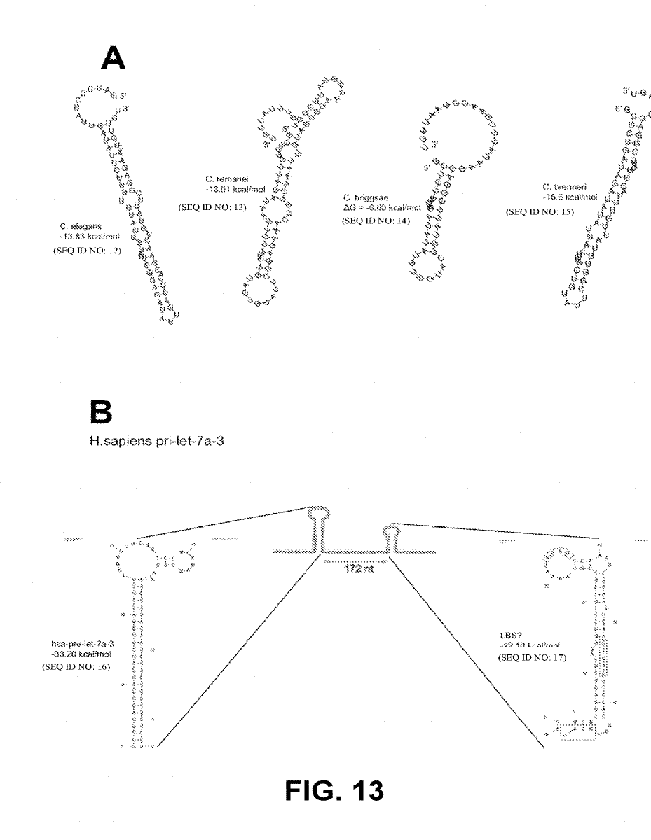

C. elegans, C. briggsae, C. remanei and C. brenneri lack GGAG motifs within the terminal loop and have elevated sequence conservation within the LBS, including at least one GGAG quadruplet in each species (FIG. 7, panel A; SEQ ID NOS: 8-11). Similarly to C. elegans, the candidate LBS sequences in other nematode species are predicted to fold into weak secondary structures. The GGAG motif in the terminal loop is present in Echinoderms, Hemichordates and Chordates. However, in all analyzed Chordate species, where several let-7 genes are present, at least one of the let-7 genes does not display the GGAG motif in their terminal loop (FIG. 7, panel B). The absence of such architecture in some members of the let-7 family suggests that LIN-28 binds elsewhere within the primary transcript, in a way similar to our findings in nematodes. In favor of this model, a predicted stem-loop structure was detected and this structure contains three GGAG motifs (SEQ ID NO: 17) 172 nucleotides downstream of the precursor stem-loop of human pri-let-7a-3 (SEQ ID NO: 16), which doesn't contain GGAG repeats, in an arrangement reminiscent of C. elegans pri-let-7 (FIG. 13).

### Validation of Additional LIN-28 Targets

Approximately 2000 C. elegans mRNAs, including those corresponding to a number of stem cell and cancer gene homologues, were among the LIN-28 molecular targets detected by CLIP.

To prioritize the list of direct LIN-28 CLIP targets, RNA-seq was utilized to identify genes with expression changes in response to lin-28 levels as described below. The set of genes that both changed expression in lin-28 mutants and were directly bound by LIN-28 were of high priority.

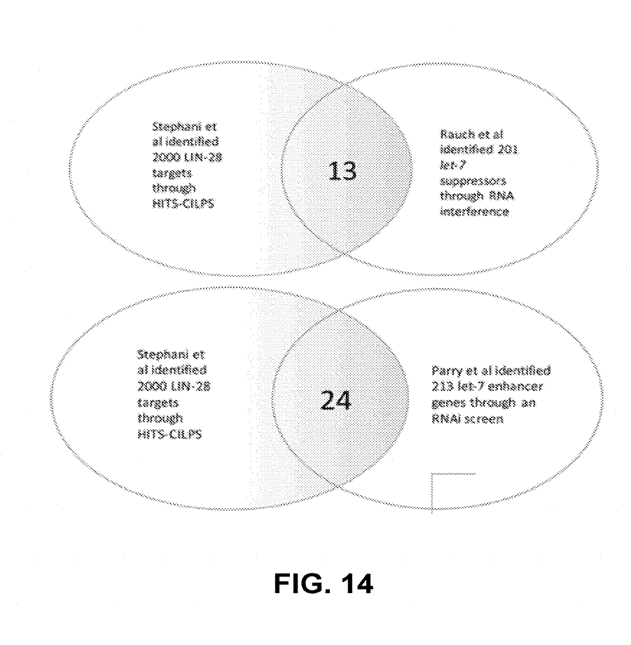

Since one of the main functions of LIN-28 was in stem cell timing and one of its main outputs was let-7 expression, lin-28 effectors which genetically interact with let-7 mutations were identified. Specifically, the CLIPseq gene list provided herein were overlapped with a set of known let-7 targets, i.e., suppressors and enhancers (FIG. 14). Specifically, these known let-7 suppressors and enhancers were previously identified from whole genome RNAi screens for suppressors of the temperature-sensitive (ts) lethality of the let-7(n2853) allele or enhancers of the weak let-7(mg179) allele.

From 201 known suppressors of let-7, 13 were identified that were also direct targets of LIN-28 binding. These genes included two known heterochronic genes, nhr-25 and kin-20, along with other genes not previously implicated in seam cell development: haf-9, let-526, rla-2, rpoa-2, iftb-1, ins-18, F55C12.1, byn-1, smc-4, ftt-2, cams-1. Of these, highest-priority for further study was kin-20, igf-1, smc-4, ftt-2 and sams-1, since their human homologues were also bound by LIN28B.

Further, from 213 known enhancers of let-7, 24 were identified that were also direct targets of LIN-28 binding: lin-28, ceh-18, F13H6.1/bcl-11, Y51A2D, 15, unc-73, rheb-1, dig-1, fin-2, lin-12, evl-14, mrck-1, mig-10, vhp-1, dyci-1, ncbp-1, let-92, cogc-4, let-526, lsy-22, C37A2.7, F01G4.6, ppk-1, rpoa-2. Of these, highest-priority for further study included lin-28, bcl-11; rheb-1, mrck-1, vhp-1, let-92, C37A2.7, F01 G4.6, ppk-1, and C37A2.7, since their human homologues were also bound by LIN28B. Among these newly identified targets, ins-18 was one of only two of the 40 insulin-like genes in C. elegans that contained a C-peptide, a feature of mammalian insulins. Smc-4 encoded a homolog of the SMC4 subunit of mitotic condensing. Ftt-2 encoded one of the two C. elegans 14-3-3 proteins. Sams-1 encoded an S-adenosyl methionine synthetase. Rheb-1 encoded a GTPase orthologous to the mammalian Rheb GTPases predicted to function as a regulator of TOR function. Mrck-1 encoded a serine/threonine-protein kinase that is orthologous to human MRCK (myotonic dystrophy kinase-related Cdc42 binding kinase) and DMPK (dystrophia myotonica-protein kinase). Vhp-1 encoded a MAP kinase phosphatase required for regulation of the KGB-1/JNK-like MAPK-mediated stress response pathway. Let-92 encoded a homolog of PP2AC, the catalytic subunit of protein phosphatase 2A (PP2A). C37A2.7 encoded an orthologue of human RPLP2 (ribosomal protein, large, P2). F01G4.6, was an orthologue of human SLC25A3 (solute carrier family 25 (mitochondrial carrier; phosphate carrier), member 3). Ppk-1 encoded a phosphatidylinositol-4-phosphate 5′ kinase. BCL11a encoded a transcription factor repressor of y-globin expression and was down-regulated by LIN28B expression.

A number of LIN-28 targets identified by CLIP were themselves regulators of gene expression, and signal transduction molecules with human homologues implicated in cancer. Accordingly, the map of the direct interactions of LIN-28 that was obtained by CLIP was expanded to encompass the global changes in the transcriptomc resulting from an absence of LIN-28. The expansion was achieved by performing RNAseq studies of lin-28 mutant animals. Specifically, measuring the total level of RNA (depleted of ribosomal RNA) by deep-sequencing had a twofold purpose: 1) it provided a reference against which to calculate enrichment in the CLIP samples; and 2) comparison of the relative abundance of LIN-28 targets defined by CLIP in total RNA extracted from wildtype and lin-28 animals provided insight on a possible mRNA (de)stabilizing role of LIN-28 binding.

It was expected that genes with altered levels would broadly fall into three categories: i) direct LIN-28 targets, which should also be present in the CLIP target pool (highest priority); ii) let-7 targets and downstream effectors, identified by the presence of let-7 complementary sequences in their 3′UTRs or previously shown to interact with let-7 mutations (FIG. 14); and iii) indirect functional targets that may not function in the seam cell pathway.

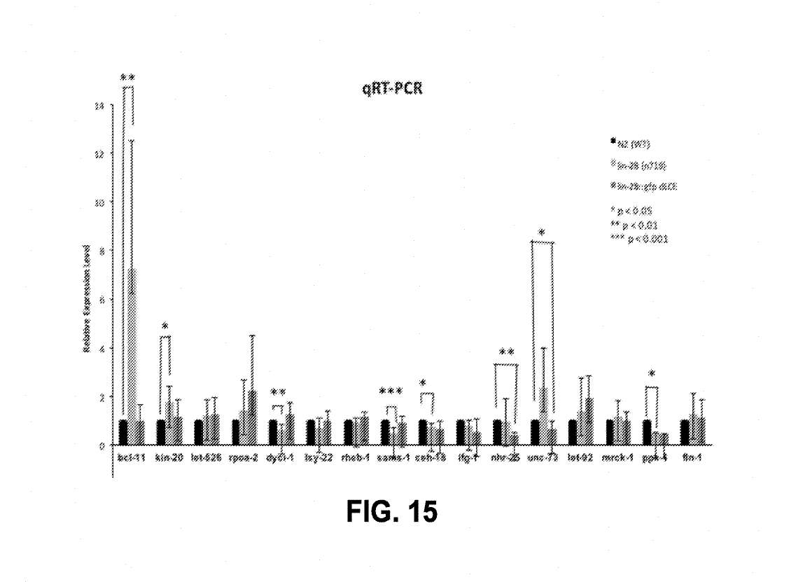

A list of genes whose expression was positively or negatively affected by the presence of lin-28 was identified by quantification and statistical analysis. A cutoff of 1.5 fold changes in gene expression and a p-value of <0.05 was used as the cut-off. Messenger RNA was isolated from three trials each of staged L1 wild-type and lin-28(n719) animals using a standard protocol. L1 stage animals were chosen because it represented a time-period when LIN-28 had been shown to function maximally during seam cell development. Differential expression analysis was performed comparing wild-type animals to similarly staged lin-28 null mutant animals. The following direct LIN-28 bound genes showed altered expression in the RNA-seq analysis: let-526; rpoa-2; bcl-11; kin-20; mrck-1; dyci-1; ifg-1; ceh-18; unc-73; nhr-25; ppk-1; iet-92; lsy-22; fln-1; sams-1; rheb-1, and these targets were validated via qRT-PCR (FIG. 15). Like KIN-20/CKIe, these genes fulfilled all the criteria of the priority workflow.

### REFERENCES

Arasu P, Wightman B, Ruvkun G. 1991. Temporal regulation of lin-14 by the inhibitoric action of two other heterochronic genes, lin-4 and lin-28. Genes Dev 5: 1825-1833.
Bailey T L, Boden M, Buske F A, Frith M, Grant C E, Clementi L, Ren J, Li W W, Noble W S. 2009. MEME Suite: tools for motif discovery and searching. Nucleic Acids Res 37: W202-W208.
Cho J, Chang H, Kwon S C, Kim B, Kim Y, Choe J, Ha M, Kim Y K, Kim V N. 2012. LIN-28A Is a Suppressor of ER-Associated Translation in Embryonic Stem Cells. Cell 151: 765-777.
Davydov I V et al. Assay for Ubiquitin Ligase Activity: High-Throughput Screen for Inhibitors of HDM, J Biomol Screen. 2004 December; 9(8):695-703
Du J. et al. Investigating the ADP-ribosyltransferase activity of sirtuins with NAD analogues and 32P-NAD, Biochemistry, 2009 Apr. 7; 48(13):2878-90
FEBS Journal, Special Issue: Protein Phosphatases: From Moecules to Networks, 280(2), 2013.
Grant C E, Bailey T L, Noble W S. 2011. FTMO: scanning for occurrences of a given motif. Bioinformatics 27: 1017-1018.
Hafner M, Max K E A, Bandaru P, Morozov P, Gerstberger S, Brown M, Molina H, Tuschl T. 2013. Identification of mRNAs bound and regulated by human LIN-28 proteins and molecular requirements for RNA recognition. RNA. http://majoumal.cshlp.org/content/early/2013/03/12/rna.036491.112 (Accessed Mar. 15, 2013).
Heo I, Joo C, Cho J, Ha M, Han J, Kim V N. 2008. Lin28 Mediates the Terminal Uridylation of let-7 Precursor MicroRNA. Molecular Cell 32: 276-284.
Huang J. An in vitro assay for the kinase activity of mTOR complex 2, Methods Mol Biol. 2012; 821:75-86
Johnson S M, Lin S Y, Slack F J. 2003. The time of appearance of the C. elegans let-7 microRNA is transcriptionally controlled utilizing a temporal regulatory element in its promoter. Dev Biol 259: 364-379.
Kishore S, Jaskiewicz L, Burger L, Hausser J, Khorshid M, Zavolan M. 2011. A quantitative analysis of CLIP methods for identifying binding sites of RNA-binding proteins. Nat Methods 8: 559-564.
McAvoy T. et al. Serine/Threonine Protein Phosphatase Assays, Curr Protoc Mol Biol. 2010 October; CHAPTER: Unit18.18
Newman M A, Thomson J M, Hammond S M. 2008. Lin-28 interaction with the Lct-7 precursor loop mediates regulated microRNA processing. RNA 14: 1539-49.
Pepper A S-R, McCane J E, Kemper K, Yeung D A, Lee R C, Ambros V, Moss E G. 2004. The C. elegans heterochronic gene lin-46 affects developmental timing at two larval stages and encodes a relative of the scaffolding protein gephyrin. Development 131: 2049-2059.
Perkel J M, Which Transcription Factor Assay Should You Use, The Scientist, July 2006.
Piskounova E, Viswanathan S R, Janas M, LaPierre R J, Daley G Q, Sliz P, Gregory R I. 2008. Determinants of microRNA processing inhibition by the developmentally regulated RNA-binding protein Lin28. J Biol Chem 283: 21310-21314.
Rather P. et al. Continuous enzymatic assay for histone lysine methyltransferases Biotechniques, 2007, 43(5): 602-passim.
Reinhart B J, Slack F J, Basson M, Pasquinelli A E, Bettinger J C, Rougvie A E, Horvitz H R, Ruvkun G. 2000. The 21-nucleotide let-7 RNA regulates developmental timing in Caenorhabditis elegans. Nature 403: 901-906.
Robinson J T, Thorvaldsdottir H, Winckler W, Guttman M, Lander E S, Getz G, Mesirov J P. 2011. Integrative genomics viewer. Nat Biotech 29: 24-26.
Singh S S et al. 2015, Targeting the PI3K/Akt signaling pathway in gastric carcinoma: A reality for personalized medicine? World J Gastroenterol. 2015 Nov. 21; 21(43): 12261-12273
Tuteja N. et al. 2004, Prokaryotic and eukaryotic DNA helicases, FEBS Journal, 271(10), 1835-48.
Viswanathan S R, Daley G Q, Gregory R T. 2008. Selective blockade of microRNA processing by Lin28. Science 320: 97-100.
Viswanathan S R, Powers J T, Einhorn W, Hoshida Y, Ng T L, Toffanin S, O'Sullivan M, Lu J, Phillips L A, Lockhart V L, et al. 2009. Lin28 promotes transformation and is associated with advanced human malignancies. Nat Genet 41: 843-848.
Web K J et al. Identification of Protein N-Terminal Methyltransferases in Yeast and Humans, Biochemistry. 2010 Jun. 29; 49(25): 5225-5235.
Wilbert M L, Huelga S C, Kapeli K, Stark T J, Liang T Y, Chen S X, Yan B Y, Nathanson J L, Hutt K R, Lovci M T, et al. 2012. LIN-28 Binds Messenger RNAs at GGAGA Motifs and Regulates Splicing Factor Abundance. Molecular Cell 48: 195-206.
Van Wynsberghe P M, Kai Z S, Massirer K B, Burton V H, Yeo G W, Pasquinelli A E. 2011. LIN-28 co-transcriptionally binds primary let-7 to regulate miRNA maturation in Caenorhabditis elegans. Nat Struct Mol Biol 18: 302-308.
Yu J, Vodyanik M A, Smuga-Otto K, Antosiewicz-Bourget J, Frane J L, Tian 5, Nie J, Jonsdottir G A, Ruotti V, Stewart R, et al. 2007. Induced Pluripotent Stem Cell Lines Derived from Human Somatic Cells. Science 318: 1917-1920.
Zhang C, Darnell R B. 2011. Mapping in vivo protein-RNA interactions at single-nucleotide resolution from HITS-CLIP data. Nat Biotechnol. http://www.ncbi.nlm.nih.gov/pubmed/21633356 (Accessed Jun. 3, 2011).
Zhu H, Shyh-Chang N, Segré AV, Shinoda G, Shah S P, Einhorn W S, Takeuchi A, Engreitz J M, Hagan J P, Kharas M G, et al. 2011. The Lin28/let-7 Axis Regulates Glucose Metabolism. Cell 147: 81-94.
Zweitzig D R, et al. Characterization of a novel DNA polymerase activity assay enabling sensitive, quantitative and universal detection of viable microbes, Nucleic Acids Res. 2012 August; 40(14): e109.
D. H. Parry, J. Xu, and G. Ruvkun, ‘A Whole-Genome Rnai Screen for C. Elegans Mirna Pathway Genes’, Curr Biol, 17 (2007), 2013-22. PMCID:PMC2211719.
M. Rausch, M. Ecsedi, H. Bartake, A. Mullner, and H. Grosshans, ‘A Genetic Interactome of the Let-7 Microrna in C. Elegans’, Dev Biol, 401 (2015), 276-86
X. C. Ding, F. J. Slack, and H. Grosshans, ‘The Let-7 Microrna Interfaces Extensively with the Translation Machinery to Regulate Cell Differentiation’, Cell Cycle. 7 (2008), 3083-90
H. Grosshans, T. Johnson, K. L. Reinert, M. Gerstein, and F. J. Slack, ‘The Temporal Patterning Microrna Let-7 Regulates Several Transcription Factors at the Larval to Adult Transition in C. Elegans’, Dev Cell, 8 (2005), 321-30
R. Graf, M. Munschauer, G. Mastrobuoni, F. Mayr, U. Heinemann, S. Kempa, N. Rajewsky, and M. Landthaler, ‘Identification of Lin28b-Bound Mrnas Reveals Features of Target Recognition and Regulation’, RNA Biol, 10 (2013), 1146-59. PMCID:PMC3849162.

TABLE 1

| Ensembl Gene ID | |
|---|---|
| of LIN-28 Targets | Associated Gene Name |
| ENSG00000004487 | KDM1A |
| ENSG00000004809 | SLC22A16 |
| ENSG00000005483 | KMT2E |
| ENSG00000005810 | MYCBP2 |
| ENSG00000005961 | ITGA2B |
| ENSG00000006744 | ELAC2 |
| ENSG00000007372 | PAX6 |
| ENSG00000007541 | PIGQ |
| ENSG00000008056 | SYN1 |
| ENSG00000008083 | JARID2 |
| ENSG00000008128 | CDK11A |
| ENSG00000008735 | MAPK8IP2 |
| ENSG00000009335 | UBE3C |
| ENSG00000010165 | METTL13 |
| ENSG00000010256 | UQCRC1 |
| ENSG00000010292 | NCAPD2 |
| ENSG00000015520 | NPC1L1 |
| ENSG00000017797 | RALBP1 |
| ENSG00000019505 | SYT13 |
| ENSG00000019995 | ZRANB1 |
| ENSG00000027075 | PRKCH |
| ENSG00000029534 | ANK1 |
| ENSG00000031081 | ARHGAP31 |
| ENSG00000032444 | PNPLA6 |
| ENSG00000033170 | FUT8 |
| ENSG00000042781 | USH2A |
| ENSG00000046889 | PREX2 |
| ENSG00000048707 | VPS13D |
| ENSG00000054179 | ENTPD2 |
| ENSG00000054356 | PTPRN |
| ENSG00000054654 | SYNE2 |
| ENSG00000058085 | LAMC2 |
| ENSG00000059145 | UNKL |
| ENSG00000059758 | CDK17 |
| ENSG00000064042 | LIMCH1 |
| ENSG00000064309 | CDON |
| ENSG00000064932 | SBNO2 |
| ENSG00000065000 | AP3D1 |
| ENSG00000065526 | SPEN |
| ENSG00000066044 | ELAVL1 |
| ENSG00000066455 | GOLGA5 |
| ENSG00000066735 | KIF26A |
| ENSG00000067836 | ROGDI |
| ENSG00000070669 | ASNS |
| ENSG00000070748 | CHAT |
| ENSG00000071282 | LMCD1 |
| ENSG00000073060 | SCARB1 |
| ENSG00000074370 | ATP2A3 |
| ENSG00000074582 | BCS1L |
| ENSG00000074590 | NUAK1 |
| ENSG00000074603 | DPP8 |
| ENSG00000074855 | ANO8 |
| ENSG00000075240 | GRAMD4 |
| ENSG00000075415 | SLC25A3 |
| ENSG00000076201 | PTPN23 |
| ENSG00000076321 | KLHL20 |
| ENSG00000077522 | ACTN2 |
| ENSG00000079785 | DDX1 |
| ENSG00000080608 | PUM3 |
| ENSG00000081237 | PTPRC |
| ENSG00000081479 | LRP2 |
| ENSG00000084090 | STARD7 |
| ENSG00000084774 | CAD |
| ENSG00000086570 | FAT2 |
| ENSG00000086598 | TMED2 |
| ENSG00000086758 | HUWE1 |
| ENSG00000087095 | NLK |
| ENSG00000087258 | GNAO1 |
| ENSG00000087263 | OGFOD1 |
| ENSG00000088298 | EDEM2 |
| ENSG00000088451 | TGDS |
| ENSG00000089163 | SIRT4 |
| ENSG00000089820 | ARHGAP4 |
| ENSG00000090565 | RAB11FIP3 |
| ENSG00000091009 | RBM27 |
| ENSG00000091129 | NRCAM |
| ENSG00000092020 | PPP2R3C |
| ENSG00000092051 | JPH4 |
| ENSG00000092621 | PHGDH |
| ENSG00000097096 | SYDE2 |
| ENSG00000099381 | SETD1A |
| ENSG00000100196 | KDELR3 |
| ENSG00000100246 | DNAL4 |
| ENSG00000100580 | TMED8 |
| ENSG00000100600 | LGMN |
| ENSG00000100632 | ERH |
| ENSG00000100889 | PCK2 |
| ENSG00000101004 | NINL |
| ENSG00000101040 | ZMYND8 |
| ENSG00000101098 | RIMS4 |
| ENSG00000101162 | TUBB1 |
| ENSG00000101444 | AHCY |
| ENSG00000101665 | SMAD7 |
| ENSG00000101825 | MXRA5 |
| ENSG00000102001 | CACNA1F |
| ENSG00000102174 | PHEX |
| ENSG00000102385 | DRP2 |
| ENSG00000102452 | NALCN |
| ENSG00000102531 | FNDC3A |
| ENSG00000102858 | MGRN1 |
| ENSG00000103047 | TANGO6 |
| ENSG00000103051 | COG4 |
| ENSG00000103266 | STUB1 |
| ENSG00000103426 | CORO7-PAM16 |
| ENSG00000103653 | CSK |
| ENSG00000104325 | DECR1 |
| ENSG00000104517 | UBR5 |
| ENSG00000104529 | EEF1D |
| ENSG00000104660 | LEPROTL1 |
| ENSG00000104695 | PPP2CB |
| ENSG00000104814 | MAP4K1 |
| ENSG00000104823 | ECH1 |
| ENSG00000104866 | PPP1R37 |
| ENSG00000104936 | DMPK |
| ENSG00000105428 | ZNRF4 |
| ENSG00000105643 | ARRDC2 |
| ENSG00000105726 | ATP13A1 |
| ENSG00000105737 | GRIK5 |
| ENSG00000105971 | CAV2 |
| ENSG00000106628 | POLD2 |
| ENSG00000106780 | MEGF9 |
| ENSG00000106789 | CORO2A |
| ENSG00000106803 | SEC61B |
| ENSG00000106927 | AMBP |
| ENSG00000107164 | FUBP3 |
| ENSG00000107242 | PIP5K1B |
| ENSG00000107281 | NPDC1 |
| ENSG00000107815 | C10orf2 |
| ENSG00000107902 | LHPP |
| ENSG00000108588 | CCDC47 |
| ENSG00000108947 | EFNB3 |
| ENSG00000109111 | SUPT6H |
| ENSG00000109689 | STIM2 |
| ENSG00000109738 | GLRB |
| ENSG00000110046 | ATG2A |
| ENSG00000110048 | OSBP |
| ENSG00000110422 | HIPK3 |
| ENSG00000110436 | SLC1A2 |
| ENSG00000110931 | CAMKK2 |
| ENSG00000111241 | FGF6 |
| ENSG00000112210 | RAB23 |
| ENSG00000112531 | QKI |
| ENSG00000112562 | SMOC2 |
| ENSG00000112578 | BYSL |
| ENSG00000112679 | DUSP22 |
| ENSG00000112818 | MEP1A |
| ENSG00000113073 | SLC4A9 |
| ENSG00000113558 | SKP1 |
| ENSG00000113810 | SMC4 |
| ENSG00000114019 | AMOTL2 |
| ENSG00000114200 | BCHE |
| ENSG00000114391 | RPL24 |
| ENSG00000114867 | EIF4G1 |
| ENSG00000115290 | GRB14 |
| ENSG00000115474 | KCNJ13 |
| ENSG00000115592 | PRKAG3 |
| ENSG00000115677 | HDLBP |
| ENSG00000116459 | ATP5F1 |
| ENSG00000116783 | TNNI3K |
| ENSG00000117480 | FAAH |
| ENSG00000117507 | FMO6P |
| ENSG00000117593 | DARS2 |
| ENSG00000117713 | ARID1A |
| ENSG00000117859 | OSBPL9 |
| ENSG00000118873 | RAB3GAP2 |
| ENSG00000119514 | GALNT12 |
| ENSG00000119688 | ABCD4 |
| ENSG00000119782 | FKBP1B |
| ENSG00000119862 | LGALSL |
| ENSG00000119915 | ELOVL3 |
| ENSG00000120729 | MYOT |
| ENSG00000120800 | UTP20 |
| ENSG00000121057 | AKAP1 |
| ENSG00000121210 | KIAA0922 |
| ENSG00000121892 | PDS5A |
| ENSG00000122406 | RPL5 |
| ENSG00000122490 | PQLC1 |
| ENSG00000123143 | PKN1 |
| ENSG00000124232 | RBPJL |
| ENSG00000124440 | HIF3A |
| ENSG00000124602 | UNC5CL |
| ENSG00000124614 | RPS10 |
| ENSG00000124664 | SPDEF |
| ENSG00000124702 | KLHDC3 |
| ENSG00000124784 | RIOK1 |
| ENSG00000125124 | BBS2 |
| ENSG00000125247 | TMTC4 |
| ENSG00000125388 | GRK4 |
| ENSG00000125630 | POLR1B |
| ENSG00000125691 | RPL23 |
| ENSG00000125977 | EIF2S2 |
| ENSG00000126391 | FRMD8 |
| ENSG00000127580 | WDR24 |
| ENSG00000127586 | CHTF18 |
| ENSG00000127884 | ECHS1 |
| ENSG00000128016 | ZFP36 |
| ENSG00000128512 | DOCK4 |
| ENSG00000128534 | LSM8 |
| ENSG00000128590 | DNAJB9 |
| ENSG00000128833 | MYO5C |
| ENSG00000128881 | TTBK2 |
| ENSG00000128965 | CHAC1 |
| ENSG00000129158 | SERGEF |
| ENSG00000129250 | KIF1C |
| ENSG00000129315 | CCNT1 |
| ENSG00000129493 | HEATR5A |
| ENSG00000129521 | EGLN3 |
| ENSG00000129596 | CDO1 |
| ENSG00000129749 | CHRNA10 |
| ENSG00000129991 | TNNI3 |
| ENSG00000130158 | DOCK6 |
| ENSG00000130529 | TRPM4 |
| ENSG00000130595 | TNNT3 |
| ENSG00000130822 | PNCK |
| ENSG00000131730 | CKMT2 |
| ENSG00000131746 | TNS4 |
| ENSG00000131914 | LIN28A |
| ENSG00000132470 | ITGB4 |
| ENSG00000132639 | SNAP25 |
| ENSG00000132763 | MMACHC |
| ENSG00000132793 | LPIN3 |
| ENSG00000132842 | AP3B1 |
| ENSG00000133115 | STOML3 |
| ENSG00000133475 | GGT2 |
| ENSG00000133597 | ADCK2 |
| ENSG00000134020 | PEBP4 |
| ENSG00000134028 | ADAMDEC1 |
| ENSG00000134551 | PRH2 |
| ENSG00000134744 | ZCCHC11 |
| ENSG00000134809 | TIMM10 |
| ENSG00000135537 | LACE1 |
| ENSG00000135723 | FHOD1 |
| ENSG00000135824 | RGS8 |
| ENSG00000136231 | IGF2BP3 |
| ENSG00000136478 | TEX2 |
| ENSG00000136542 | GALNT5 |
| ENSG00000136628 | EPRS |
| ENSG00000136750 | GAD2 |
| ENSG00000136937 | NCBP1 |
| ENSG00000137274 | BPHL |
| ENSG00000137571 | SLCO5A1 |
| ENSG00000137766 | UNC13C |
| ENSG00000138101 | DTNB |
| ENSG00000138138 | ATAD1 |
| ENSG00000138246 | DNAJC13 |
| ENSG00000138326 | RPS24 |
| ENSG00000138468 | SENP7 |
| ENSG00000138709 | LARP1B |
| ENSG00000138801 | PAPSS1 |
| ENSG00000139116 | KIF21A |
| ENSG00000139160 | METTL20 |
| ENSG00000139517 | LNX2 |
| ENSG00000139726 | DENR |
| ENSG00000140506 | LMAN1L |
| ENSG00000140553 | UNC45A |
| ENSG00000140829 | DHX38 |
| ENSG00000140990 | NDUFB10 |
| ENSG00000141367 | CLTC |
| ENSG00000141543 | EIF4A3 |
| ENSG00000141551 | CSNK1D |
| ENSG00000141564 | RPTOR |
| ENSG00000141646 | SMAD4 |
| ENSG00000141946 | ZIM3 |
| ENSG00000142233 | NTN5 |
| ENSG00000142676 | RPL11 |
| ENSG00000143183 | TMCO1 |
| ENSG00000143190 | POU2F1 |
| ENSG00000143322 | ABL2 |
| ENSG00000143376 | SNX27 |
| ENSG00000143473 | KCNH1 |
| ENSG00000143499 | SMYD2 |
| ENSG00000143947 | RPS27A |
| ENSG00000144406 | UNC80 |
| ENSG00000144410 | CPO |
| ENSG00000144589 | STK11IP |
| ENSG00000144821 | MYH15 |
| ENSG00000144908 | ALDH1L1 |
| ENSG00000145214 | DGKQ |
| ENSG00000145730 | PAM |
| ENSG00000145907 | G3BP1 |
| ENSG00000145916 | RMND5B |
| ENSG00000146223 | RPL7L1 |
| ENSG00000146414 | SHPRH |
| ENSG00000146555 | SDK1 |
| ENSG00000147044 | CASK |
| ENSG00000147416 | ATP6V1B2 |
| ENSG00000147576 | ADHFE1 |
| ENSG00000147647 | DPYS |
| ENSG00000147799 | ARHGAP39 |
| ENSG00000148396 | SEC16A |
| ENSG00000148908 | RGS10 |
| ENSG00000149043 | SYT8 |
| ENSG00000149091 | DGKZ |
| ENSG00000149428 | HYOU1 |
| ENSG00000149781 | FERMT3 |
| ENSG00000150961 | SEC24D |
| ENSG00000151224 | MAT1A |
| ENSG00000151475 | SLC25A31 |
| ENSG00000151490 | PTPRO |
| ENSG00000151611 | MMAA |
| ENSG00000152217 | SETBP1 |
| ENSG00000152332 | UHMK1 |
| ENSG00000153071 | DAB2 |
| ENSG00000153132 | CLGN |
| ENSG00000153179 | RASSF3 |
| ENSG00000153707 | PTPRD |
| ENSG00000154227 | CERS3 |
| ENSG00000154258 | ABCA9 |
| ENSG00000154358 | OBSCN |
| ENSG00000154889 | MPPE1 |
| ENSG00000155657 | TTN |
| ENSG00000156030 | ELMSAN1 |
| ENSG00000156052 | GNAQ |
| ENSG00000156885 | COX6A2 |
| ENSG00000157219 | HTR5A |
| ENSG00000157483 | MYO1E |
| ENSG00000158008 | EXTL1 |
| ENSG00000158186 | MRAS |
| ENSG00000158856 | DMTN |
| ENSG00000158859 | ADAMTS4 |
| ENSG00000159363 | ATP13A2 |
| ENSG00000159409 | CELF3 |
| ENSG00000159459 | UBR1 |
| ENSG00000159496 | RGL4 |
| ENSG00000159753 | RLTPR |
| ENSG00000159899 | NPR2 |
| ENSG00000160299 | PCNT |
| ENSG00000160326 | SLC2A6 |
| ENSG00000160460 | SPTBN4 |
| ENSG00000160606 | TLCD1 |
| ENSG00000160972 | PPP1R16A |
| ENSG00000161057 | PSMC2 |
| ENSG00000161395 | PGAP3 |
| ENSG00000161681 | SHANK1 |
| ENSG00000161956 | SENP3 |
| ENSG00000161960 | EIF4A1 |
| ENSG00000162631 | NTNG1 |
| ENSG00000162643 | WDR63 |
| ENSG00000162676 | GFI1 |
| ENSG00000162733 | DDR2 |
| ENSG00000162949 | CAPN13 |
| ENSG00000163207 | IVL |
| ENSG00000163291 | PAQR3 |
| ENSG00000163406 | SLC15A2 |
| ENSG00000163581 | SLC2A2 |
| ENSG00000163673 | DCLK3 |
| ENSG00000163900 | TMEM41A |
| ENSG00000164068 | RNF123 |
| ENSG00000164073 | MFSD8 |
| ENSG00000164129 | NPY5R |
| ENSG00000164172 | MOCS2 |
| ENSG00000164318 | EGFLAM |
| ENSG00000164329 | PAPD4 |
| ENSG00000164506 | STXBP5 |
| ENSG00000164695 | CHMP4C |
| ENSG00000164733 | CTSB |
| ENSG00000165269 | AQP7 |
| ENSG00000165392 | WRN |
| ENSG00000165795 | NDRG2 |
| ENSG00000165917 | RAPSN |
| ENSG00000166226 | CCT2 |
| ENSG00000166266 | CUL5 |
| ENSG00000166441 | RPL27A |
| ENSG00000166816 | LDHD |
| ENSG00000166866 | MYO1A |
| ENSG00000167100 | SAMD14 |
| ENSG00000167283 | ATP5L |
| ENSG00000167526 | RPL13 |
| ENSG00000167550 | RHEBL1 |
| ENSG00000167658 | EEF2 |
| ENSG00000167693 | NXN |
| ENSG00000167769 | ACER1 |
| ENSG00000167792 | NDUFV1 |
| ENSG00000168071 | CCDC88B |
| ENSG00000168394 | TAP1 |
| ENSG00000168619 | ADAM18 |
| ENSG00000168781 | PPIP5K1 |
| ENSG00000169026 | MFSD7 |
| ENSG00000169067 | ACTBL2 |
| ENSG00000169359 | SLC33A1 |
| ENSG00000169599 | NFU1 |
| ENSG00000169764 | UGP2 |
| ENSG00000170027 | YWHAG |
| ENSG00000170296 | GABARAP |
| ENSG00000170684 | ZNF296 |
| ENSG00000171155 | C1GALT1C1 |
| ENSG00000171723 | GPHN |
| ENSG00000171914 | TLN2 |
| ENSG00000172671 | ZFAND4 |
| ENSG00000172766 | NAA16 |
| ENSG00000172794 | RAB37 |
| ENSG00000172869 | DMXL1 |
| ENSG00000172901 | LVRN |
| ENSG00000172954 | LCLAT1 |
| ENSG00000174016 | FAM46D |
| ENSG00000174231 | PRPF8 |
| ENSG00000174611 | KY |
| ENSG00000174672 | BRSK2 |
| ENSG00000175198 | PCCA |
| ENSG00000175329 | ISX |
| ENSG00000175766 | EIF4E1B |
| ENSG00000175806 | MSRA |
| ENSG00000176142 | TMEM39A |
| ENSG00000176946 | THAP4 |
| ENSG00000176978 | DPP7 |
| ENSG00000177105 | RHOG |
| ENSG00000177192 | PUS1 |
| ENSG00000177200 | CHD9 |
| ENSG00000177350 | RPL13AP3 |
| ENSG00000177479 | ARIH2 |
| ENSG00000177600 | RPLP2 |
| ENSG00000177733 | HNRNPA0 |
| ENSG00000178425 | NT5DC1 |
| ENSG00000178804 | H1FOO |
| ENSG00000178928 | TPRX1 |
| ENSG00000179134 | SAMD4B |
| ENSG00000179241 | LDLRAD3 |
| ENSG00000179632 | MAF1 |
| ENSG00000179869 | ABCA13 |
| ENSG00000180251 | SLC9A4 |
| ENSG00000180264 | ADGRD2 |
| ENSG00000181143 | MUC16 |
| ENSG00000181222 | POLR2A |
| ENSG00000181381 | DDX60L |
| ENSG00000181588 | MEX3D |
| ENSG00000182179 | UBA7 |
| ENSG00000182389 | CACNB4 |
| ENSG00000182544 | MFSD5 |
| ENSG00000182551 | ADI1 |
| ENSG00000182676 | PPP1R27 |
| ENSG00000182871 | COL18A1 |
| ENSG00000183020 | AP2A2 |
| ENSG00000183048 | SLC25A10 |
| ENSG00000183114 | FAM43B |
| ENSG00000183248 | PRR36 |
| ENSG00000183780 | SLC35F3 |
| ENSG00000184304 | PRKD1 |
| ENSG00000184428 | TOP1MT |
| ENSG00000184508 | HDDC3 |
| ENSG00000184611 | KCNH7 |
| ENSG00000184845 | DRD1 |
| ENSG00000185344 | ATP6V0A2 |
| ENSG00000185532 | PRKG1 |
| ENSG00000186009 | ATP4B |
| ENSG00000186575 | NF2 |
| ENSG00000186642 | PDE2A |
| ENSG00000186716 | BCR |
| ENSG00000186795 | KCNK18 |
| ENSG00000186919 | ZACN |
| ENSG00000187231 | SESTD1 |
| ENSG00000187240 | DYNC2H1 |
| ENSG00000187546 | AGMO |
| ENSG00000188107 | EYS |
| ENSG00000188167 | TMPPE |
| ENSG00000188467 | SLC24A5 |
| ENSG00000188886 | ASTL |
| ENSG00000189037 | DUSP21 |
| ENSG00000189079 | ARID2 |
| ENSG00000189350 | FAM179A |
| ENSG00000196169 | KIF19 |
| ENSG00000196177 | ACADSB |
| ENSG00000196459 | TRAPPC2 |
| ENSG00000196465 | MYL6B |
| ENSG00000196632 | WNK3 |
| ENSG00000196730 | DAPK1 |
| ENSG00000196850 | PPTC7 |
| ENSG00000197157 | SND1 |
| ENSG00000197415 | VEPH1 |
| ENSG00000197451 | HNRNPAB |
| ENSG00000197892 | KIF13B |
| ENSG00000197969 | VPS13A |
| ENSG00000198216 | CACNA1E |
| ENSG00000198554 | WDHD1 |
| ENSG00000198586 | TLK1 |
| ENSG00000198707 | CEP290 |
| ENSG00000198758 | EPS8L3 |
| ENSG00000198838 | RYR3 |
| ENSG00000198843 | SELT |
| ENSG00000198863 | RUNDC1 |
| ENSG00000203740 | METTL11B |
| ENSG00000203818 | HIST2H3PS2 |
| ENSG00000203857 | HSD3B1 |
| ENSG00000203995 | ZYG11A |
| ENSG00000204178 | TMEM57 |
| ENSG00000204316 | MRPL38 |
| ENSG00000204574 | ABCF1 |
| ENSG00000204644 | ZFP57 |
| ENSG00000205126 | ACCSL |
| ENSG00000205143 | ARID3C |
| ENSG00000205318 | GCNT6 |
| ENSG00000205869 | KRTAP5-1 |
| ENSG00000205978 | NYNRIN |
| ENSG00000212724 | KRTAP2-3 |
| ENSG00000213079 | SCAF8 |
| ENSG00000213445 | SIPA1 |
| ENSG00000213563 | C8orf82 |
| ENSG00000213741 | RPS29 |
| ENSG00000213889 | PPM1N |
| ENSG00000213901 | SLC23A3 |
| ENSG00000213930 | GALT |
| ENSG00000214491 | SEC14L6 |
| ENSG00000215218 | UBE2QL1 |
| ENSG00000215262 | KCNU1 |
| ENSG00000215305 | VPS16 |
| ENSG00000216490 | IFI30 |
| ENSG00000216671 | CCNYL3 |
| ENSG00000219491 | TPT1P8 |
| ENSG00000221955 | SLC12A8 |
| ENSG00000223802 | CERS1 |
| ENSG00000224569 | RP11-341G5.2 |
| ENSG00000224776 | RPSAP50 |
| ENSG00000226400 | CTD-2269E23.2 |
| ENSG00000227825 | SLC9A7P1 |
| ENSG00000229119 | CTB-63M22.1 |
| ENSG00000231305 | RP11-723O4.2 |
| ENSG00000231500 | RPS18 |
| ENSG00000234125 | EEF1GP8 |
| ENSG00000240771 | ARHGEF25 |
| ENSG00000241360 | PDXP |
| ENSG00000243244 | STON1 |
| ENSG00000243543 | WFDC6 |
| ENSG00000243725 | TTC4 |
| ENSG00000243789 | JMJD7 |
| ENSG00000244694 | PTCHD4 |
| | BCL11a |

1. A method of identifying an agent for treating LIN-28-expressing cancer, comprising:
providing a LIN-28 target, which is optionally selected from the LIN-28 targets in Table 1,
testing candidate agents for modulating the activity or expression of the LIN-28 target, and
selecting a candidate agent that modulates the activity or expression of the LIN-28 target.

2. The method of claim 1, wherein a LIN-28 target is selected by inhibiting the expression of targets from Table 1 in a cell line that requires LIN-28 for growth.

3. The method of claim 2, wherein at least 10 targets from Table 1 are evaluated for their impact on LIN-28-dependent cell growth or impact on let-7 activity in said cell line.

4. The method of any one of claims 1 to 3, wherein the LIN-28 target is a kinase.

5. The method of claim 4, wherein the LIN-28 target is CDK11A, CDK17, NUAK1, NLK, PCK2, CSK, MAP4K1, DMPK, PTP5K1B, HIPK3, CAMKK2, RIOK1, GRK4, TTBK2, ADCK2, CSNK1D/E, ABL2, CASK, UHMK1, DCLK3WNK3, DAPK1, or TLK1,

6. The method of any one of claims 4 to 5, wherein the candidate agents are tested for modulation of the activity of the target in a kinase assay.

7. The method of any one of claims 1 to 3, wherein the LIN-28 target is a methyltransferase.

8. The method of claim 7, wherein the LIN-28 target is KMT2E or METTL11B.

9. The method of claim 7 or 8, wherein the candidate agents are tested for modulation of the activity of the target in a methyltransferase assay.

10. The method of any one of claims 1 to 3, wherein the LIN-28 target is a phosphatase.

11. The method of claim 10, wherein the LIN-28 target is PTPRN, PTPN23, PPP2R3C, PPP2CB, PPP1R37, PPP1R16A, PDXP, or SETD1A.

12. The method of claim 10 or 11, wherein the candidate agents are tested for modulation of the activity of the target in a phosphatase assay.

13. The method of any one of claims 1 to 3, wherein the LIN-28 target is a transcription factor or helicase.

14. The method of claim 13, wherein the LIN-28 target is PAX6, DDX1, SMAD7, ARTD1A, SMAD4, POU2F1, WRN, CHD9, ARTD2, ARTD3C, BCL11A or JARTD2.

15. The method of claim 13 or 14, wherein the candidate agents are tested for modulation of the activity of the target in a transcription, polynucleotide-binding, or helicase assay.

16. The method of any one of claims 1 to 3, wherein the LIN-28 target is a methylase, demethylase, or acetylase.

17. The method of claim 16, wherein the LIN-28 target is SIRT4.

18. The method of claim 16 or 17, wherein the candidate agents are tested for modulation of the activity of the target in a methylase, demethylase, or acetylase assay.

19. The method of any one of claims 1 to 3, wherein the LIN-28 target is a DNA or RNA polymerase.

20. The method of claim 19, wherein the LIN-28 target is POLD2 or POLR2A.

21. The method of claim 20, wherein the candidate agents are tested for modulation of the activity of the target in a DNA or RNA polymerase assay.

22. The method of any one of claims 1 to 3, wherein the LIN-28 target is a E3 ubiquitin-protein ligase.

23. The method of claim 22, wherein the E3 ubiquitin-protein ligase is SKP1 or ARIH2.

24. The method of claim 22 or 23, wherein the candidate agents are tested for modulation of the activity of the target in a ubiquitin-protein ligase assay.

25. The method of any one of claims 1 to 3, wherein the LIN-28 target is in the mTOR pathway.

26. The method of claim 25, wherein the LIN-28 target is RPTOR.

27. The method of claim 25 or 26, wherein the candidate agents are tested for modulation of the activity of the mTOR pathway.

28. The method of any one of claims 1 to 3, wherein the LIN-28 target is a protease.

29. The method of claim 28, wherein the LIN-28 target is ADAMTS4 or ADAM18.

30. The method of claim 29, wherein the candidate agents are tested for modulation of the activity of the target in a protease assay.

31. The method of any one of claims 1 to 3, wherein the LIN-28 target is a phosphodiesterase.

32. The method of claim 31, wherein the LIN-28 target is PDE2.

33. The method of any one of claims 1 to 3, wherein the candidate agents are antisense polynucleotides, which optionally comprise the motif GGAG or CTCC, an siRNA or antisense molecule targeting the mRNA corresponding to the gene, or an agent which optionally mimics the action of a miRNA selected from a let-7 family member or miR-229 family.

34. The method of claim 33, wherein the candidate agents are assayed for modulation of expression or abundance of the LIN-28 target in a cell.

35. The method of any one of claims 1 to 34, wherein the modulation of activity or expression is confirmed in an animal model.

36. The method of claim 35, wherein the selected candidate agent is tested against a LIN-28 expressing cancer in an animal model.

37. The method of claims 1 to 36, wherein the candidate agent is derivatized, and tested for enhanced activity against the LIN-28 target in vitro or in vivo.

38. The method of any one of claims 1 to 37, wherein the selected agent is formulated as a pharmaceutically-acceptable composition.

39. A method for making a pharmaceutical composition useful for treating LIN-28-expressing cancer, comprising:
identifying a candidate agent according to any one of claims 1 to 36; and
formulating said agent or a derivative thereof as a pharmaceutical composition.

40. A method for treating a subject having cancer, comprising:
administering the composition made according to the method of claims 1 to 38 to said subject.

41. The method of claim 40, wherein the cancer is a LIN-28 positive or LIN-28-overexpressing cancer.

42. The method of claim 40, wherein a biopsy of the subject's tumor is tested for expression of LIN-28 and/or a LIN-28 target from Table 1.


SEQUENCE LISTING (17 sequences)

SEQ ID NO: 1 - RNA, 16 nt, Artificial Sequence
  Synthetic Sequence
  misc_binding (1)..(1): 5AmMC6 linked to adenine nucleotide
agggaggacgaugcgg

SEQ ID NO: 2 - RNA, 20 nt, Artificial Sequence
  Synthetic Sequence
  misc_binding (1)..(1): Phosphate binds to the nucleotide
  misc_binding (20)..(20): Pmn binds to the nucleotide
gugucagucacuuccagcgg

SEQ ID NO: 3 - DNA, 52 bp, Artificial Sequence
  Synthetic Sequence
aatgatacggcgaccaccgactatggatacttagtcagggaggacgatgcgg

SEQ ID NO: 4 - DNA, 41 bp, Artificial Sequence
  Synthetic Sequence
caagcagaagacggcatacgaccgctggaagtgactgacac

SEQ ID NO: 5 - DNA, 32 bp, Artificial Sequence
  Synthetic Sequence
ctatggatacttagtcagggaggacgatgcgg

SEQ ID NO: 6 - RNA, 64 nt, Artificial Sequence
  Synthetic Sequence
ugagguaguagguuguauaguuuggaauauuaccaccggugaacuaugcaauuuucuaccuuac

SEQ ID NO: 7 - RNA, 62 nt, Artificial Sequence
  Synthetic Sequence
gauauucuuuuguacuguauucggagauauuguuucacgaacuguauucggagaacuguugu

SEQ ID NO: 8 - DNA, 72 bp, Caenorhabditis elegans
gatccctattgatattcttttgtactgtattcggagatattgtttcacgaactgtattcggagaactgttgt

SEQ ID NO: 9 - DNA, 79 bp, Caenorhabditis remanei
gctctgttagataatttttactgtactgtattcggagaaacgttctaattgtactggaactgtattcggtgtttattgt

SEQ ID NO: 10 - DNA, 54 bp, Caenorhabditis briggsae
gctctgttagatatattttgtactgtattcggaggaatatttgaacctaattgt

SEQ ID NO: 11 - DNA, 61 bp, Caenorhabditis brenneri
gctctgatagatatattactgtactgtattcggtgtattgtgtactgtattcggaggtcgt

SEQ ID NO: 12 - RNA, 72 nt, Caenorhabditis elegans
gaucccuauugauauucuuuuguacugauuucggagauauuguuucacgaacuguauucggagaacuguugu

SEQ ID NO: 13 - RNA, 79 nt, Caenorhabditis remanei
gcucuguuagauaauuuuuuacuguacuguauucggagaaacguucuaauuguacuggaacuguauucggguuuauugu

SEQ ID NO: 14 - RNA, 54 nt, Caenorhabditis briggsae
gcucuguuagauauauuuuguacuguauucggaggaauauuugaagguaauugu

SEQ ID NO: 15 - RNA, 61 nt, Caenorhabditis brenneri
gcucugauagauauauuacucuacuguauucgguguauuguguacuguauucggaggucgu

SEQ ID NO: 16 - RNA, 74 nt, Homo sapiens
gggugagguaguagguuguauaguuuggggcucugcccugcuaugggauaacuauacaaucuacugucuuuccu

SEQ ID NO: 17 - RNA, 71 nt, Homo sapiens
ucucugggucucaguuuccuccucuguaaaauggaggcaaaugaggauggaaggagaugcaguguggagca